(19) United States
(12) Patent Application Publication
(10) Pub. No.: US 2024/0175876 A1
(43) Pub. Date: May 30, 2024

(54) PERIPLASMIC LIGAND TRAPPING SYSTEM

(71) Applicant: UNIVERSITY OF MIAMI, Miami, FL (US)

(72) Inventors: Daniel Isom, Miami, FL (US); Jacob Rowe, Miami, FL (US)

(21) Appl. No.: 18/522,521

(22) Filed: Nov. 29, 2023

Related U.S. Application Data
(60) Provisional application No. 63/385,395, filed on Nov. 29, 2022.

(51) Int. Cl. G01N 33/68; C07K 14/395; C12N 1/18; G01N 21/64; G01N 33/53

(52) U.S. Cl. CPC G01N 33/6845; C07K 14/395; C12N 1/185; G01N 21/6486; G01N 33/53

(57) ABSTRACT

Disclosed herein are engineered yeast cells, systems, and methods for screening for a ligand that modulates a receptor function and for determining functional consequences of receptor-ligand binding.

### CROSS REFERENCE TO RELATED APPLICATIONS

This application claims the benefit of U.S. Provisional Patent Application Ser. No. 63/385,395 filed Nov. 29, 2022, which is expressly incorporated herein by reference.

### STATEMENT REGARDING FEDERALLY SPONSORED RESEARCH

This invention was made with government support under R35GM119518 awarded by the National Institutes of Health. The government has certain rights in the invention.

### REFERENCE TO A SEQUENCE LISTING

A computer readable form of the Sequence Listing is filed with this application by electronic submission and is incorporated into this application by reference in its entirety. The Sequence Listing is contained in the file created on Nov. 28, 2023, having the file name “11348-054US1_ST26” and is 68,924 bytes in size.

### FIELD

This disclosure relates to reagents and methods for assessing receptor binding as well as determining functional consequences of receptor-ligand binding, particularly by using yeast cells genetically engineered to express peptide-, protein-, and nanobody-based ligands that are trapped between the yeast cell wall and cell membrane, i.e., periplasmic space.

### BACKGROUND

The yeast Saccharomyces cerevisiae is an established model for the heterologous study of human transmembrane receptors such as G protein-coupled receptors (GPCRs) and receptor tyrosine kinases (RTKs). Through genetic engineering, the yeast model has become a robust and highly scalable drug-discovery platform for identifying small molecule pharmacological ligands. By contrast, efforts to study and discover genetically encoded peptide ligands, protein ligands, and pharmacological tools selective for protein conformation and function remains a major challenge for both human- and yeast-based discovery platforms. The new technologies described in this application address this important unmet need.

Existing technologies display proteins on the outside of the yeast cell wall to discover interactions between a protein bait (e.g., nanobody) and target (e.g., receptor or viral component). In these approaches, selection is based solely on bait and target binding using a pull-down assay with the purified protein target immobilized on a bead. Although these methods have identified many new protein-protein interactions, they suffer from several critical limitations that have hindered their further development. These include 1) the need to over-express and purify sufficient amounts of a protein target, which may not be possible, 2) non-specific binding of the purified target to the assay beads, which creates the need for several rounds of enrichment, and most critically 3) little to no information regarding the functional selectivity of the bait-target interaction.

Thus, there remains a need in the art to develop methods for assessing receptor binding as well as determining functional consequences of receptor-ligand binding for human proteins in yeast cells.

### SUMMARY

Provided herein are systems and methods for assessing receptor binding as well as determining functional consequences of receptor-ligand binding in yeast cells that both utilize and overcome the limitations of the cell wall as a situs for displaying receptors and any other proteins of interest with sufficient and necessary periplasmic accessibility and spatial exclusivity. The methods disclosed herein describe a cell wall anchoring protein for displaying peptide-, protein-, and nanobody-based ligands in the periplasmic space (i.e., the space between cell wall and cell membrane) rather than on the yeast surface. In this way, the periplasmic-displayed ligand is presented to the ligand-binding interface of the receptor, where it is capable of modulating receptor function for the purposes of studying ligand-receptor biology or identifying new ligand-receptor or protein-protein interactions via high-throughput screening experiments.

In some aspects, disclosed herein is a yeast periplasmic ligand-trapping yeast display system comprising: a plurality of yeast cells, wherein each cell comprises:
a recombinant expression construct encoding:
   a ligand;
   a cell-wall anchoring protein, wherein the ligand is fused to the cell-wall anchoring protein; and
   a signal sequence, wherein the signal sequence promotes secretion of the one or more ligands to the yeast cell periplasmic space; and
an inducible reporter.

In some embodiments, the cell-wall anchoring protein comprises an N terminus projected in the periplasmic space, and wherein the ligand is fused to the N terminus of the cell-wall anchoring protein.

In some embodiments, the yeast cells are Saccharomyces cerevisiae. In some embodiments, the ligand is a genetically encoded peptide, protein, or nanobody.

In some embodiments, the signal sequence comprises a sequence at least 80% identical to SEQ ID NO:20, SEQ ID NO:21, SEQ ID NO:22, SEQ ID NO:23, SEQ ID NO:24, SEQ ID NO:25, or SEQ ID NO:26. In some embodiments, the signal sequence comprises SEQ ID NO:21.

In some embodiments, the cell-wall anchoring protein comprises a sequence at least 80% identical to SEQ ID NO:13, SEQ ID NO:14, SEQ ID NO:15, SEQ ID NO:16, SEQ ID NO:17, SEQ ID NO:18, or SEQ ID NO:19. In some embodiments, the cell-wall anchoring protein comprises SEQ ID NO:13.

In some embodiments, the inducible reporter produces a fluorescent protein. In some embodiments, the fluorescent protein is mTurqoise2.

In some embodiments, the inducible reporter is dependent on activation of a receptor.

In some aspects, disclosed herein is a method of screening for a ligand that modulates a receptor function, comprising:
providing a yeast periplasmic ligand-trapping yeast display system comprising: a plurality of yeast cells, wherein each cell comprises:
   a recombinant expression construct encoding:
      a ligand;
      a cell-wall anchoring protein, wherein the ligand is fused to the cell-wall anchoring protein; and
      a signal sequence, wherein the signal sequence promotes secretion of the one or more ligands to the yeast cell periplasmic space; and
   an inducible reporter;
expressing the ligand fused to the cell-wall anchoring protein;
measuring the expression of the inducible reporter; and
determining if the ligand modulates a receptor function in comparison to a reference control.

In some embodiments, the yeast cells are Saccharomyces cerevisiae. In some embodiments, the ligand is a genetically encodable peptide, protein, or nanobody.

In some embodiments, the signal sequence comprises a sequence at least 80% identical to SEQ ID NO:20, SEQ ID NO:21, SEQ ID NO:22, SEQ ID NO:23, SEQ ID NO:24, SEQ ID NO:25, or SEQ ID NO:26. In some embodiments, the signal sequence comprises SEQ ID NO:21.

In some embodiments, the cell-wall anchoring protein comprises a sequence at least 80% identical to SEQ ID NO:13, SEQ ID NO:14, SEQ ID NO:15, SEQ ID NO:16, SEQ ID NO:17, SEQ ID NO:18, or SEQ ID NO:19. In some embodiments, the cell-wall anchoring protein comprises SEQ ID NO:13.

In some embodiments, the inducible reporter produces a fluorescent protein. In some embodiments, the fluorescent protein is mTurqoise2.

In some embodiments, the inducible reporter is dependent on activation of a receptor.

### BRIEF DESCRIPTION OF THE DRAWINGS

The disclosure will be better understood and features, aspects, and advantages other than those set forth above will become apparent when consideration is given to the following detailed description thereof. Such detailed description refers to the following drawings.

FIGS. 1A-1B illustrate periplasmic display for genetically encodable ligands in the ENTRAP platform. FIG. 1A is a schematic diagram illustrating a cell wall protein (dark gray), i.e., anchor, whose N-terminus is oriented in the periplasm. FIG. 1B shows the genetic components of optimized trapping technology that includes an N-terminal αPrePro signal sequence (SEQ ID NO:1) for secretion, any genetically encodable ligand, and a C-terminal cell wall anchor containing amino acids 23-238 of the native yeast protein Ccw14 (SEQ ID NO:2).

FIG. 2 is a schematic diagram of the periplasmic ligand trapping technology for functional studies of membrane receptors in the ENTRAP platform. The periplasmic ligand trapping platform comprises a cell wall anchoring protein (1) used for anchoring and displaying protein ligands in the periplasm (2). This allows the ligand to pharmacologically modulate a membrane-bound receptor (3), which interacts with and regulates defined intracellular proteins (4) upon receptor activation. Activation of these signaling components is then monitored via a fluorescent reporter (5), providing a functional readout of ligand-receptor interactions.

FIGS. 3A-3B show experiments for the activation of different ENTRAP platforms. FIG. 3A illustrates (left) and reports (right) experimental data for the activation of the somatostatin 5 receptor (SSTR5) by its cell-wall anchored peptide agonist SRIF-14. The anchored peptide agonist is trapped and displayed in the yeast periplasm (1) enabling it to activate SSTR5 (2) and stimulate signaling via four different Ga proteins. FIG. 3B illustrates (left) and reports (right) experimental data for the activation of chemokine receptor 4 (CXCR4) by its cell-wall anchored chemokine agonist CXCL12a (3). The anchored chemokine agonist is trapped and displayed in the yeast periplasm enabling it to activate CXCR4 and stimulate signaling via two Ga proteins. In this platform, GPCR activation leads to expression of a fluorescent transcriptional reporter, mTurquoise2, which is used to quantify signaling in relative fluorescence units (RFU). Data are the mean±SD (n=4).

FIGS. 4A-4B show experiments for the antagonism of a GPCR using the ENTRAP platform. FIG. 4A is a schematic diagram of yeast strains expressing GPCR CXCR4 (1), the cognate protein agonist CXCL12a, and the CXCR4-specific nanobody CA4139 attached to the periplasmic display protein Ccw14 (2). FIG. 4B shows CXCR4 signaling profiles with galactose-inducible expression of CXCL12a alone (gray line) and CXCL12a expression with periplasmic displayed nanobody CA4139 (black line, dashed). Results from expression of CXCR4 only, i.e., with no CXCL12a or Nb, are shown as negative control (white). Data are the mean±SD (n=4).

FIGS. 5A-5D show experiments for GPCR modulation by intracellular nanobodies using the modified ENTRAP platform. FIG. 5A is a schematic of yeast strains expressing the angiotensin receptor 1 (AGTR1) (1), its cognate peptide agonist AGT-II, and AGTR1-specific intracellular nanobody (2). FIG. 5B shows AGTR1 signaling profiles with galactose-inducible expression of AGT-II alone (black line), AGT-II expression with intracellular non-specific nanobody (Nb) (gray line), and AGT-II expression with AGTR1-specific intracellular nanobodies (dashed) AT110, FIG. 5C. AT1101103 (Left), and AT110i1 (Right). Results from expression of AGTR1 only, i.e., with no AGT-II or Nb, are shown as negative control (white). Data are the mean±SD (n=4). FIG. 5D. Fold change in AGTR1 activation in the presence of intracellular nanobodies described in FIG. 5B-FIG. 5C. Data is relative to AGTR1 activation with AGT-II alone.

FIGS. 6A-6B illustrate the process of building and screening synthetic nanobody libraries that can be profiled against GPCRs using the ENTRAP platform. GPCRs. FIG. 6A. Models of the three particularly advantageous consensus nanobody scaffolds with their CDR3 loops (1). The ENTRAP platform can be implemented for extra- and intracellular nanobody libraries using yeast expression plasmids (FIG. 6A) or CRISPR editing (FIG. 6B) exclusively, and in combination.

### DETAILED DESCRIPTION

For the purposes of promoting an understanding of the disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Terminology

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by a person of ordinary skill in the art to which this disclosure belongs.

### Systems and Methods

In some aspects, disclosed herein is a yeast periplasmic ligand-trapping yeast display system comprising: a plurality of yeast cells, wherein each cell comprises:
a recombinant expression construct encoding:
   a ligand;
   a cell-wall anchoring protein, wherein the ligand is fused to the cell-wall anchoring protein; and
   a signal sequence, wherein the signal sequence promotes secretion of the one or more ligands to the yeast cell periplasmic space; and
an inducible reporter.

In some embodiments, the cell-wall anchoring protein is fused to the N terminus of the cell-wall anchoring protein. In some embodiments, the cell-wall anchoring protein is fused to the C terminus of the cell-wall anchoring protein.

In some embodiments, the cell-wall anchoring protein comprises an N terminus projected in the periplasmic space, and wherein the ligand is fused to the N terminus of the cell-wall anchoring protein.

In some aspects, disclosed herein is a yeast periplasmic ligand-trapping yeast display system comprising: a plurality of yeast cells, wherein each cell comprises:
a recombinant expression construct encoding:
   a ligand;
   a cell-wall anchoring protein having an N terminus projected in the periplasmic space, and wherein the ligand is fused to the N terminus of the cell-wall anchoring protein; and
   a signal sequence, wherein the signal sequence promotes secretion of the one or more ligands to the yeast cell periplasmic space; and
an inducible reporter.

In some embodiments, the yeast cells are Saccharomyces cerevisiae. In some embodiments, the ligand is a genetically encodable peptide, protein, or nanobody.

In some embodiments, the signal sequence comprises a sequence at least 60% identical (for example, at least 60%, at least 65%, at least 70%, at least 75%, at least 80%, at least 85%, at least 90%, or at least 95%) to SEQ ID NO:20, SEQ ID NO:21, SEQ ID NO:22, SEQ ID NO:23, SEQ ID NO:24, SEQ ID NO:25, or SEQ ID NO:26. In some embodiments, the signal sequence comprises a sequence at least 80% identical to SEQ ID NO:20, SEQ ID NO:21, SEQ ID NO:22, SEQ ID NO:23, SEQ ID NO:24, SEQ ID NO:25, or SEQ ID NO:26. In some embodiments, the signal sequence comprises SEQ ID NO:20, SEQ ID NO:21, SEQ ID NO:22, SEQ ID NO:23, SEQ ID NO:24, SEQ ID NO:25, or SEQ ID NO:26.

In some embodiments, the signal sequence comprises a sequence at least 60% identical (for example, at least 60%, at least 65%, at least 70%, at least 75%, at least 80%, at least 85%, at least 90%, or at least 95%) to SEQ ID NO:21. In some embodiments, the signal sequence comprises SEQ ID NO:21.

In some embodiments, the signal sequence comprises a sequence at least 60% identical (for example, at least 60%, at least 65%, at least 70%, at least 75%, at least 80%, at least 85%, at least 90%, or at least 95%) to SEQ ID NO:24. In some embodiments, the signal sequence comprises SEQ ID NO:24.

In some embodiments, the signal sequence comprises a sequence at least 60% identical (for example, at least 60%, at least 65%, at least 70%, at least 75%, at least 80%, at least 85%, at least 90%, or at least 95%) to SEQ ID NO:26. In some embodiments, the signal sequence comprises SEQ ID NO:26.

In some embodiments, the cell-wall anchoring protein comprises a sequence at least 60% identical (for example, at least 60%, at least 65%, at least 70%, at least 75%, at least 80%, at least 85%, at least 90%, or at least 95%) to SEQ ID NO:13, SEQ ID NO:14, SEQ ID NO:15, SEQ ID NO:16, SEQ ID NO:17, SEQ ID NO:18, or SEQ ID NO:19. In some embodiments, the cell-wall anchoring protein comprises a sequence at least 80% identical to SEQ ID NO:13, SEQ ID NO:14, SEQ ID NO:15, SEQ ID NO:16, SEQ ID NO:17, SEQ ID NO:18, or SEQ ID NO:19. In some embodiments, the cell-wall anchoring protein comprises SEQ ID NO: 13, SEQ ID NO:14, SEQ ID NO:15, SEQ ID NO:16, SEQ ID NO:17, SEQ ID NO: 18, or SEQ ID NO:19.

In some embodiments, the cell-wall anchoring protein comprises a sequence at least 60% identical (for example, at least 60%, at least 65%, at least 70%, at least 75%, at least 80%, at least 85%, at least 90%, or at least 95%) to SEQ ID NO:13. In some embodiments, the cell-wall anchoring protein comprises SEQ ID NO:13.

In some embodiments, the cell-wall anchoring protein comprises a sequence at least 60% identical (for example, at least 60%, at least 65%, at least 70%, at least 75%, at least 80%, at least 85%, at least 90%, or at least 95%) to SEQ ID NO:14. In some embodiments, the cell-wall anchoring protein comprises SEQ ID NO:14.

In some embodiments, the cell-wall anchoring protein comprises a sequence at least 60% identical (for example, at least 60%, at least 65%, at least 70%, at least 75%, at least 80%, at least 85%, at least 90%, or at least 95%) to SEQ ID NO:15. In some embodiments, the cell-wall anchoring protein comprises SEQ ID NO:15.

In some embodiments, the cell-wall anchoring protein comprises a sequence at least 60% identical (for example, at least 60%, at least 65%, at least 70%, at least 75%, at least 80%, at least 85%, at least 90%, or at least 95%) to SEQ ID NO:17. In some embodiments, the cell-wall anchoring protein comprises SEQ ID NO:17.

In some embodiments, the inducible reporter produces a fluorescent protein. In some embodiments, the fluorescent protein is mTurqoise2.

In some embodiments, the inducible reporter is dependent on activation of a receptor.

In some aspects, disclosed herein is a method of screening for a ligand that modulates a receptor function, comprising:
providing a yeast periplasmic ligand-trapping yeast display system comprising: a plurality of yeast cells, wherein each cell comprises:
   a recombinant expression construct encoding:
      a ligand;
      a cell-wall anchoring protein, wherein the ligand is fused to the cell-wall anchoring protein; and
      a signal sequence, wherein the signal sequence promotes secretion of the one or more ligands to the yeast cell periplasmic space; and
   an inducible reporter;
expressing the ligand fused to the cell-wall anchoring protein;
measuring the expression of the inducible reporter; and
   determining if the ligand modulates a receptor function in comparison to a reference control.

In some aspects, disclosed herein is a method of screening for a ligand that modulates a receptor function, comprising:
providing a yeast periplasmic ligand-trapping yeast display system comprising: a plurality of yeast cells, wherein each cell comprises:
   a recombinant expression construct encoding:
      a ligand;
      a cell-wall anchoring protein having an N terminus projected in the periplasmic space, and wherein the ligand is fused to the N terminus of the cell-wall anchoring protein; and
      a signal sequence, wherein the signal sequence promotes secretion of the one or more ligands to the yeast cell periplasmic space; and
   an inducible reporter;
expressing the ligand fused to the N terminus of the cell-wall anchoring protein;
measuring the expression of the inducible reporter; and
determining if the ligand modulates a receptor function in comparison to a reference control.

In some embodiments, the yeast cells are Saccharomyces cerevisiae. In some embodiments, the ligand is a genetically encodable peptide, protein, or nanobody.

In some embodiments, the signal sequence comprises a sequence at least 60% identical (for example, at least 60%, at least 65%, at least 70%, at least 75%, at least 80%, at least 85%, at least 90%, or at least 95%) to SEQ ID NO:20, SEQ ID NO:21, SEQ ID NO:22, SEQ ID NO:23, SEQ ID NO:24, SEQ ID NO:25, or SEQ ID NO:26. In some embodiments, the signal sequence comprises a sequence at least 80% identical to SEQ ID NO:20, SEQ ID NO:21, SEQ ID NO:22, SEQ ID NO:23, SEQ ID NO:24, SEQ ID NO:25, or SEQ ID NO:26. In some embodiments, the signal sequence comprises SEQ ID NO:20, SEQ ID NO:21, SEQ ID NO:22, SEQ ID NO:23, SEQ ID NO:24, SEQ ID NO:25, or SEQ ID NO:26.

In some embodiments, the signal sequence comprises a sequence at least 60% identical (for example, at least 60%, at least 65%, at least 70%, at least 75%, at least 80%, at least 85%, at least 90%, or at least 95%) to SEQ ID NO:21. In some embodiments, the signal sequence comprises SEQ ID NO:21.

In some embodiments, the signal sequence comprises a sequence at least 60% identical (for example, at least 60%, at least 65%, at least 70%, at least 75%, at least 80%, at least 85%, at least 90%, or at least 95%) to SEQ ID NO:24. In some embodiments, the signal sequence comprises SEQ ID NO:24.

In some embodiments, the signal sequence comprises a sequence at least 60% identical (for example, at least 60%, at least 65%, at least 70%, at least 75%, at least 80%, at least 85%, at least 90%, or at least 95%) to SEQ ID NO:26. In some embodiments, the signal sequence comprises SEQ ID NO:26.

In some embodiments, the cell-wall anchoring protein comprises a sequence at least 60% identical (for example, at least 60%, at least 65%, at least 70%, at least 75%, at least 80%, at least 85%, at least 90%, or at least 95%) to SEQ ID NO:13, SEQ ID NO:14, SEQ ID NO:15, SEQ ID NO:16, SEQ ID NO:17, SEQ ID NO:18, or SEQ ID NO:19. In some embodiments, the cell-wall anchoring protein comprises a sequence at least 80% identical to SEQ ID NO:13, SEQ ID NO:14, SEQ ID NO:15, SEQ ID NO:16, SEQ ID NO:17, SEQ ID NO:18, or SEQ ID NO:19. In some embodiments, the cell-wall anchoring protein comprises SEQ ID NO:13, SEQ ID NO:14, SEQ ID NO:15, SEQ ID NO:16, SEQ ID NO:17, SEQ ID NO:18, or SEQ ID NO:19.

In some embodiments, the cell-wall anchoring protein comprises a sequence at least 60% identical (for example, at least 60%, at least 65%, at least 70%, at least 75%, at least 80%, at least 85%, at least 90%, or at least 95%) to SEQ ID NO:13. In some embodiments, the cell-wall anchoring protein comprises SEQ ID NO:13.

In some embodiments, the cell-wall anchoring protein comprises a sequence at least 60% identical (for example, at least 60%, at least 65%, at least 70%, at least 75%, at least 80%, at least 85%, at least 90%, or at least 95%) to SEQ ID NO:14. In some embodiments, the cell-wall anchoring protein comprises SEQ ID NO: 14.

In some embodiments, the cell-wall anchoring protein comprises a sequence at least 60% identical (for example, at least 60%, at least 65%, at least 70%, at least 75%, at least 80%, at least 85%, at least 90%, or at least 95%) to SEQ ID NO:15. In some embodiments, the cell-wall anchoring protein comprises SEQ ID NO:15.

In some embodiments, the cell-wall anchoring protein comprises a sequence at least 60% identical (for example, at least 60%, at least 65%, at least 70%, at least 75%, at least 80%, at least 85%, at least 90%, or at least 95%) to SEQ ID NO:17. In some embodiments, the cell-wall anchoring protein comprises SEQ ID NO:17.

In some embodiments, the inducible reporter produces a fluorescent protein. In some embodiments, the fluorescent protein is mTurqoise2.

In some embodiments, the inducible reporter is dependent on activation of a receptor.

In some embodiments, the method allows screening of a nanobody library. In some embodiments, the method allows screening of a peptide library. In some embodiments, the method allows screening of a small molecule library.

In some embodiments, the systems and methods use yeast engineered for the functional expression of human G-protein coupled receptors (GPCRs), the utility of the anchored ligand design using known peptide, protein, and nanobody ligands is demonstrated, whose effects on receptor functionality are directly assessed inter alia via fluorescent reporting of receptor activation using microplate readers, fluorescence activated cell sorting, high-content microscopy, and any other methods that provide quantification of the fluorescent reporter. The platform can also be used to detect intracellular ligands and nanobodies selective for specific receptor conformations and multimeric states.

In some embodiments, the systems and methods use yeast engineered for the functional expression of a Chemokine Receptor (for example, the C—X—C Motif Chemokine Receptor 4 (CXCR4)). In some embodiments, the systems and methods use yeast engineered for the functional expression of Angiotensin II Receptor Type I (AGTR1).

In some embodiments, herein is described a novel platform, ENTRAP, that overcomes these barriers to identify and select conformation-specific ligands and nanobodies for GPCRs based not only on binding, but also on functional and pharmacological outcomes, including receptor agonism, inverse agonism, antagonism, allosteric modulation and conformational blocking.

In some embodiments, the systems and methods use yeast engineered for the functional expression of human receptor tyrosine kinases (RTKs).

Also provided herein is a method of identifying receptor function, the method comprising: providing a plurality of yeast cells, wherein each cell comprises, one or more ligands displayed in the periplasm of the yeast cells, wherein the one or more ligands are anchored to the cell wall by a cell-wall anchoring protein; and wherein the ligand or ligands are capable of ligand-receptor binding to a transmembrane receptor expressed at the yeast cell wall surface; wherein the transmembrane receptor is bound to an intracellular protein, wherein the intracellular protein is encoded with an inducible reporter; wherein the inducible reporter is fluorescent protein, and wherein expression of the inducible reporter is dependent on activation of the transmembrane receptor by the one or more ligands.

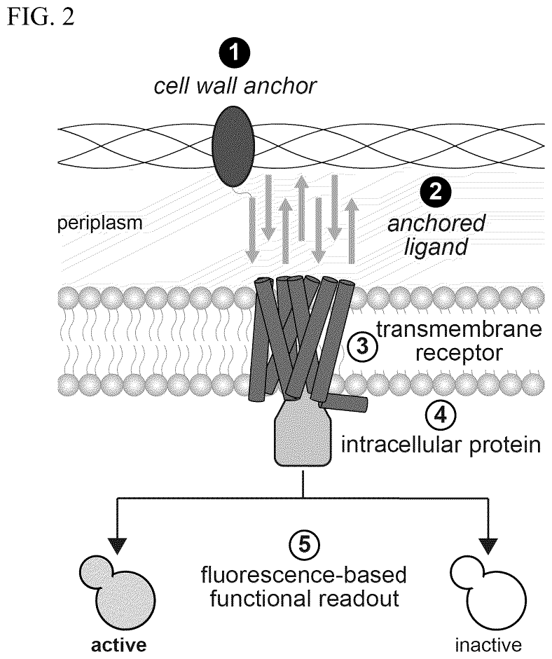

The ligand trapping technology disclosed herein was designed with compatibility for yeast-based platforms used to study human transmembrane receptors and other cell surface proteins. Displaying peptides, proteins, or nanobodies in the periplasm near the receptor's ligand-binding site (i.e., orthosteric site) permits ligand-receptor interactions to occur. As shown in FIG. 2, interactions resulting in receptor activation led to subsequent activation of intracellular proteins measured via a fluorescent reporter. Yeast-based systems using the disclosed periplasmic ligand display uniquely provides a functional assessment of ligand-receptor interactions, as opposed to prior yeast display systems that lack functional readouts and solely assess ligand binding to purified receptors.

Combining these technologies created a pharmacological screening platform with broad applications. First, the ligand trapping technology allows engineering of autocrine signaling systems, where endogenous peptide/protein ligands can be displayed in yeast strains harboring their cognate receptors. These strains can then be used for screening any chemical library to identify potential antagonists, providing a unique advantage over most other screening platforms commonly biased towards agonist discovery. These strains can also be used for screening libraries of purified peptides and proteins to identify potential antagonists.

This combinatorial platform also allows screening of gene-based libraries encoding a variety of peptide, protein, or nanobody ligands and libraries. The libraries can comprise DNA or RNA, and can comprise a genetically encodeable ligand for GPCRs or other receptors that can be expressed to the yeast membrane. This approach resulted in a major time savings and financial advantage over conventional screening approaches for these ligand types, which required initial ligand purification with limited yields. Additionally, many of these ligands cannot be overexpressed and purified from conventional sources or traverse the yeast cell wall when added exogenously, limiting the use of yeast screening platforms prior to the periplasmic ligand trapping technology disclosed herein.

Lastly, in some embodiments, each individual cell contains a single genetically encoded receptor and ligand. These genetic components serve as identity barcodes for deconvolution of functional ligand-receptor interactions in pooled experiments. As a consequence, pooling of both ligand and receptor libraries can be performed simultaneously, with functional interactions being identified merely through fluorescence- and sequence-based methods.

As used herein the term “recombinant expression construct” will be understood by a person having ordinary skill in the art to mean a product of genetic engineering techniques for introducing into a cell a nucleic acid encoding a protein of interest, particularly a protein heterologous to the cell into which the recombinant expression construct is introduced.

The term “G Protein-Coupled Receptor” or “GPCR” refers to any member of the large family of transmembrane receptors that typically function to bind molecules outside the cell and activate inside signal transduction pathways, ultimately inducing one or more cellular responses. G protein-coupled receptors are found only in eukaryotes, including yeast and animals.

Binding and activation of a GPCR typically involves signal transduction pathways including the cAMP signal pathway and the phosphatidylinositol signal pathway. When a ligand binds to the GPCR it causes a conformational change in the GPCR, which allows it to act as a guanine nucleotide exchange factor (GEF). The GPCR can then activate an associated G-protein by exchanging its bound GDP for a GTP. The G-protein's a subunit, together with the bound GTP, can then dissociate from the β and γ subunits to further affect intracellular signaling proteins or target functional proteins directly depending on the a subunit type (Gαs, Gαi/o, Gαq/11, Gα12/13).

In some embodiments, the intracellular protein comprises a sequence at least 60% identical (for example, at least 60%, at least 65%, at least 70%, at least 75%, at least 80%, at least 85%, at least 90%, or at least 95%) to SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, or SEQ ID NO: 12. In some embodiments, the cell-wall anchoring protein comprises a sequence at least 80% identical to SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO: 12. In some embodiments, the cell-wall anchoring protein comprises SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, or SEQ ID NO: 12.

All GPCRs share a common structure and mechanism of signal transduction. Generally, GPCRs can be grouped into 6 classes based on sequence homology and functional similarity: Class A (or 1) (Rhodopsin-like), Class B (or 2) (Secretin receptor family), Class C (or 3) (Metabotropic glutamate/pheromone), Class D (or 4) (Fungal mating pheromone receptors), Class E (or 5) (Cyclic AMP receptors), Class F (or 6) (Frizzled/Smoothened). Many G protein-coupled receptors are involved in detection of endogenous ligands (e.g., hormones, growth factors, etc.).

In some embodiments, the GPCR is localized to the cell membrane. In some embodiments, the GPCR gene is integrated into the cell's genome. In some embodiments, the inducible reporter is integrated into the cell's genome.

In some embodiments, the target domain gene comprises a “barcode” or a unique sequence. The barcode is used to uniquely identify or distinguish the target domain. The barcode may be of any suitable length for unambiguously identifying the target domain gene. The length of the barcode sequence is not critical and may be of any length sufficient to distinguish the barcode sequence from other barcode sequences. In some embodiments, the target domain gene is heterologous to the yeast system and represents a unique DNA sequence that can be identified by quantitative polymerase chain reaction, NanoString, sequencing, and similar methods.

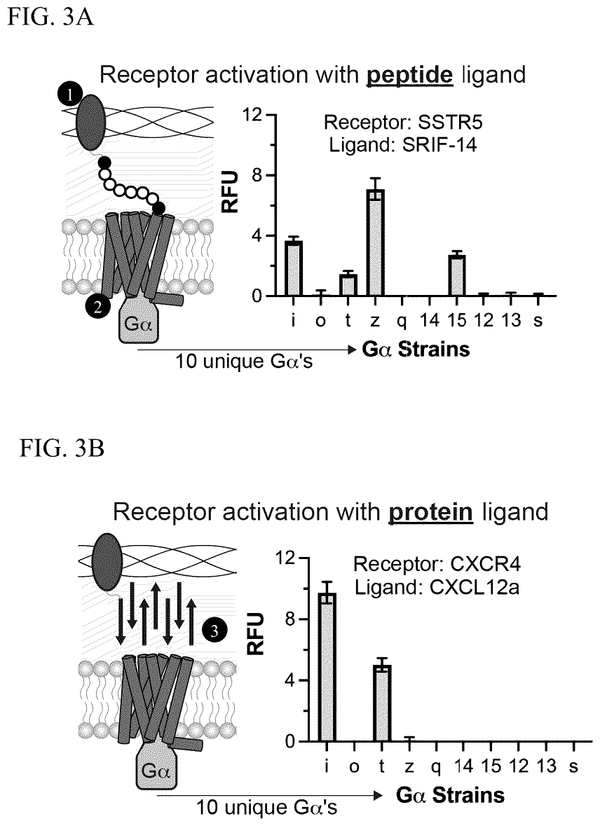

In some embodiments, the reporter is induced by signal transduction upon activation of the GPCR (shown, e.g. in FIG. 3B). In some embodiments, the reporter comprises one or more of a CAMP response element (CRE), a nuclear factor of activated T-cells response element (NFAT-RE), serum response element (SRE), and serum response factor response element (SRF-RE). In some embodiments, the reporter is a transcriptional reporter such as mTurquoise2 (mTq2). In some embodiments, the mTq2 reporter replaces the pheromone-responsive gene open reading frame in the cell.

In some embodiments, the cells are yeast cells selected from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans.

In some embodiments, the cells can comprise any other yeast species that has a cell wall and pheromone-sensing pathway. In some embodiments, the cells can comprise yeast from the evolutionary kingdom Fungi and division Ascomycota that have a cell wall and conserved pheromone-sensing pathway.

In some embodiments, the cell-wall anchoring protein is any of Ccw14₂₃₋₂₃₈, Sed1₁₉₋₃₃₈, Flo1₁₄₉₆₋₁₅₃₇, Flo5₂₅₋₁₀₇₅, Suc2₂₀₋₅₃₂, Ecm3₃₂₀₋₄₂₉, and Yps1₂₂₋₅₆₉. In some embodiments, the cell-wall anchoring protein is Ccw14₂₃₋₂₃₈. Ccw 14₂₃₋₂₃₈ is localized to the inner cell wall in yeast.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The Examples set forth herein incorporate and rely on certain experimental and preparatory methods and techniques preformed as exemplified herein.

### Example 1: Materials and Methods

### Bacterial Strains and Growth Media

NEB® 5-alpha Competent Escherichia coli (E. coli), a derivative of the DH5α strain, were used for all cloning experiments and general plasmid propagation. Initial selection of E. coli was performed at 37° C. on Lysogeny Broth (LB) agar medium supplemented with 100 mg/L carbenicillin: 10.0 g/L tryptone, 5.0 g/L yeast extract, 5.0 g/L sodium chloride (Sigma), 0.1 g/L sodium hydroxide, 18.0 g/L Bacto agar. Select transformants were then grown at 37° C., shaking at 200 rpm, in LB medium supplemented with 50 mg/L carbenicillin.

### Yeast Strains and Growth Media

All yeast strains used are derivatives of BY4741 (MATa his3Δ1 leu2Δ0 met15Δ0 ura3Δ0), are listed in Table 1 and have been described previously (Kapolka et al., 2020, Proc. Natl. Acad. Sci. USA 117: 13117-13126). Specifically, these strains are derivatives of the DI3Δfig1Δ::mTq2 P1 model strain (BY4741 far1Δ0 sst2Δ0 ste2Δ0 fig1Δ0::mTq2 X-2:PTEF1a-UnTS-TCYC1b) containing humanized Ga proteins.

Prior to transformation or other experimentation, yeast cells were grown at 30° C. on Yeast extract Peptone Dextrose (YPD) agar medium: 10.0 g/L yeast extract (RPI), 20.0 g/L peptone (RPI), 20.0 g/L dextrose (RPI), 15.0 g/L Bacto agar (RPI). Select colonies were propagated at 30° C., static or shaking at 200 rpm, in liquid cultures of YPD or pH-adjusted Synthetic Complete Dextrose low fluorescence medium (SCD Screening Medium) as specified comprising 5.0 g/L ammonium sulfate, 0.79 g/L CSM supplement mixture, 20.0 g/L dextrose, 9.76 g/L MES monohydrate, 8.72 g/L potassium phosphate dibasic, 1.70 g/L low fluorescence yeast nitrogen base without amino acids, folic acid, and riboflavin, pH adjusted with potassium hydroxide or hydrochloric acid.

For yeast transformations, transformants were initially selected at 30° C. for three days on SCD dropout agar medium comprising 5.0 g/L ammonium sulfate, 20.0 g/L dextrose, 1.70 g/L yeast nitrogen base without amino acids and ammonium sulfate, 0.1 g/L sodium hydroxide, 15.0 g/L Bacto agar, and 0.69 g/L of Complete Supplement Mixture (CSM)-Leu or 0.67 g/L of CSM-Leu-Ura. Select transformants were propagated at 30° C., static or shaking at 200 rpm, in liquid cultures of SCD dropout medium.

### Data Acquisition

A CLARIOstar multimode microplate reader was used for collecting all microplate-based absorbance (A600) and fluorescence data with the following instrument settings: absorbance (excitation 600 nm; 22 flashes/well), mTq2-based fluorescence (excitation 430/10 nm, dichroic LP 458 nm, emission 482/16 nm; 10 flashes/well; bottom read). FIGS. 3A-3B, 4A-4B, and 5A-5D show data derived from GPCR assays that were collected every 1 h for 15 h, all data from each replicate was fit in GraphPad Prism 8.3.0 (San Diego, CA) to generate slope and intercept values, which were used to extrapolate fluorescence to a standardized A₆₀₀ value of 1.0 as described previously (Rowe et al., 2021, J. Biol. Chem. 296: 100167).

### Plasmids and Construct Design

All plasmids used and constructed throughout this study were purified using EZ Plasmid Miniprep Kit and sequences validated via Sanger sequencing.

Plasmids used for testing yeast anchoring proteins were constructed by amplifying the respective genes via PCR from the BY4741 genome, starting immediately downstream of the signal peptide cleavage site, except for Flo1₁₄₉₆₋₁₅₃₇, which contains only the 42 C-terminal amino acids of Flo1, synthesized as a gBlock (IDT) as described previously (Rowe et al., 2020, J. Biol. Chem. 295: 8262-8271). Amplicons were cloned into pYEplac181 αPreₙₐₜ using NEBuilder® HiFi DNA Assembly Master Mix (NEB; catalog no. E2621L), where αPreₙₐₜ is the pre α-factor signal sequence. The gene for the fluorescent protein mTurquoise2, mTq2, was then inserted between αPreₙₐₜ and the anchor protein-encoding gene in similar fashion, with yeast codon-optimized mTq2 being sourced from Addgene (#86424). Sequences for all anchor proteins tested are provided in Table 2.

Six unique sequences were selected for comparison with αPreₙₐₜ used initially for secretion, which included: the native pre-pro α-factor signal sequence (αPreProₙₐₜ), three optimized pre-pro α-factor variants (αPreProᵥ₂ (Aza et al., 2021, Cell Mol Life Sci 78: 3691-3707)), αPreProᵥ₃ (Id.), and αPreProᵥ₄ (Rakestraw et al., 2009, Biotechnol Bioeng 103: 1192-1201)), the signal sequence native to the S. cerevisiae oligosaccharyltransferase complex alpha subunit (Ost1; YJL002C), and a chimeric pre-pro α-factor sequence where the pre α-factor sequence has been replaced with that from Ost1 (OstlaPro) (Bcan et al., 2022, Nat Commun. 13: 2882; Fitzgerald et al., 2014, Microbial Cell Factories 13: 125). Complete signal sequences were first generated via Assembly PCR using 2-4 overlapping oligonucleotides 40-100 bp long. Once assembled, amplicons were directly cloned into pYEplac181 as described above, followed by cloning of mTq2 downstream of the specified signal sequence. Sequences for all signal sequences tested are provided in Table 3.

GPCRs via periplasmic ligand display (FIG. 3A-3B): GPCR genes SSTR5 and CXCR4 were amplified from Presto Tango library (Krocze et al., 2015, Nat Struct Mol Biol 22: 362-369) and cloned into pYEplac195 using NEBuilder® HiFi DNA Assembly Master Mix for testing autocrine activation. The CXCR4 mutation N119A was subsequently introduced for compatibility with the yeast model (Rosenberg et al., 2019, Cell Chem Biol 26: 662-673 e667) using a modified site-directed mutagenesis approach described previously (Rowe et al., 2021, J. Biol. Chem. 296: 100167). Inverse PCR was performed using a 5′-phosphorylated reverse oligonucleotide accompanied by a forward oligonucleotide containing a 3-bp 5′ overhang encoding the amino acid alanine. The linearized PCR product was digested with DpnI, ligated, and sequence validated following transformation and purification. Plasmids expressing periplasmic-displayed ligands, SRIF-14 and CXCL12a genes were ordered as yeast codon-optimized gBlocks (IDT, Coralville, IA) and cloned into pYEplac181 αPreProₙₐₜ-Ccw14 to obtain pYEplac181 αPreProₙₐₜ-ligand-Ccw14 constructs.

A novel vector pYEplac181M2 was designed, which provided both constitutive (TEF1A promoter) and inducible (GAL1 promoter) expression similarly to that described previously (Vickers et al., 2013, Microbial Cell Factories 12: 96). This was done by PCR-amplifying the GAL1 promoter and ADH1 terminator sequences from pYDS649HM (McMahon et al., 2018, Nat Struct Mol Biol 25: 289-296) and simultaneously cloning into pYEplac181. Then, pYEplac181M2_αPreProₙₐₜ-CXCL12a was created by simultaneously cloning αPreProₙₐₜ and CXCL12a genes downstream of the GAL1 promoter using HiFi DNA Assembly. A similar approach was used for creating pYEplac181M2_αPreProₙₐₜ-AGTII, but αPreProₙₐₜ-AGTII was first assembled via Assembly PCR using a yeast codon-optimized (IDT) AGT-II sequence and subsequently cloned into pYEplac181M2 under GAL1 regulation. Lastly, periplasmic-displayed nanobody CA4139 (αPreProₙₐₜ-CA4139-Ccw14₂₃₋₂₃₈) or intracellular nanobodies AT110, AT110i103, AT110i1, and BV025 (McMahon et al., 2018, Nat Struct Mol Biol 25: 289-296) were cloned into pYEplac181M2 αPreProₙₐₜ-CXCL12a or pYEplac181M2_αPreProₙₐₜ-AGTII, respectively, under TEF1A regulation using HiFi DNA Assembly.

### Bacterial Transformations

A Mix & Go E. coli Transformation Kit (Zymo Research, Irvine, CA) and associated protocol was used to make competent bacterial cells. Competent cells were stored as 210-uL aliquots in sterile 1.5-mL Eppendorf tubes at −80° ° C. for long-term storage. For transformation, cells were thawed on ice, gently mixed, and 50 uL aliquoted to sterile 1.5-mL round-bottom Eppendorf tubes on ice. Desired DNA (1-5 uL) was added to cells, gently flicked 4-5 times, and placed on ice for 15 min. Cell mixtures were then heat shocked at 42° C. for 30 sec, recovered on ice for 5 minutes, and 900 uL room-temperature Super Optimal broth with Catabolite repression (SOC) was added prior to rotating at 37° ° C. for 45 min. Once transformation was complete, 100 uL transformant cells were plated onto LB agar medium supplemented with appropriate antibiotics.

### Yeast Transformations

Prior to transformation, select yeast colonies were picked from YPD agar medium into 5 mL YPD liquid medium in sterile 50-mL conical tubes and grown to saturation overnight. Cultures were then diluted 1:50 in 5-15 mL YPD in sterile 50-mL conical tubes and grown for 2.5-3 h to A₆₀₀ 0.4-1.0. Cells were made chemically competent following a standard lithium acetate protocol as described previously (Kapolka et al., 2020, Proc. Natl. Acad. Sci. USA 117: 13117-13126).

50 uL competent yeast cells were added to DNA transformation mixtures containing 175 uL PEG mix (400 g/L PEG-3350 dissolved in LiOAc mix), 5 uL salmon sperm DNA (stored on ice after initial boiling at 100° ° C. for 10 minutes) (Thermo Fisher) and 150-200 ng plasmid DNA (150 ng for single plasmid transformations; 100 ng of each plasmid for co-transforming two plasmids) in sterile 1.5-mL Eppendorf tubes. Mixtures were vortexed, incubated at room temperature for 30 minutes, spiked with 12 uL DMSO (Sigma), and heat shocked at 42° C. for 15 minutes. Transformant yeast cells were then centrifuged (5000 g for 1 minute), harvested, and resuspended in 200-400 uL YPD liquid medium. 35-50 uL or 100 uL resuspended cells were plated onto small (22-mm, 12-well) or large (100-mm) petri dishes, respectively, containing appropriate SCD dropout agar medium and placed at 30° ° C. for three days.

### Autocrine GPCR Activation Via Periplasmic Ligand Display

For autocrine activation of receptors SSTR5 and CXCR4 with periplasmic-displayed ligands SRIF-14 and CXCL12a, respectively, the 10 yeast strains harboring humanized Ga proteins (DI DCyFIR P1 I-DI DCyFIR P1 S (2); see Table 1) were co-transformed with pYEplac195 receptor and pYEplac181 αPreProₙₐₜ-ligand-Ccw 14.

Four transformant yeast colonies per strain were picked from SCD-Ura-Leu agar medium into 1 mL SCD-Ura-Leu medium in sterile 96-well deep-well blocks, covered with porous film, shaken on a MixMate microplate shaker (1200 rpm for 30 seconds), and grown at 30° C. static for 18-19 h. Cells were then resuspended via shaking (1500 rpm for 1 minute), A₆₀₀ measured, and used to prepare 80 uL cultures normalized to A₆₀₀ 0.1 in SCD Screening Medium pH 7.0 in a sterile black 384-well clear-bottom plate. 384-well plate was then centrifuged (3000 g for 1 minute), shaken (2000 rpm for 30 seconds), and placed in a microplate reader pre-warmed to 30° C. and programmed to collect A₆₀₀ and fluorescence (gain 1200) measurements every 1 h for 15 h.

### CXCR4 Modulation Via Periplasmic Display of Nanobody CA4139

The yeast strain harboring the humanized Gαi protein (DI DCyFIR P1 I) was co-transformed with pYEplac195 CXCR4 and one of the following pYEplac181M2 constructs: pYEplac181M2_αPreProₙₐₜ-CXCL12a (CXCL12a (Gal induced)), pYEplac181M2 αPreProₙₐₜ-CA4139-Ccw14_αPreProₙₐₜ-CXCL12a (CXCL12a+Nb CA4139), or empty pYEplac181M2 (neg ctrl) for testing nanobody-based modulation of CXCR4 using periplasm-displayed nanobody CA4139. For each pYEplac181M2 construct listed, the components under regulation of the constitutive TEF1A promoter are listed first, where applicable, followed by components under the inducible GAL1 promoter (i.e., pYEplac181M2 component_{constitutive}_component_{inducible}).

Four transformant yeast colonies per transformation were picked from SCD-Ura-Leu agar medium into 1 mL SCD-Ura-Leu medium in a sterile 96-well deep-well block, covered with porous film, shaken on a MixMate microplate shaker (1200 rpm for 30 seconds), and grown at 30° C. static for 21 h. Cells were then resuspended via shaking (1500 rpm for 1 minute), A₆₀₀ measured, and used to prepare 1.5 mL cultures normalized to A₆₀₀ 0.2 in low-glucose SCD-Ura-Leu medium (1.9% galactose, 0.1% glucose) in a sterile 96-well deep-well block. Block was covered with porous film and grown at 30° C. static for 21 h. Cells were then resuspended again and A₆₀₀ measured. Using a Biomek NXP liquid-handling robot, the volume of cells needed for preparing 100-uL cultures normalized to A₆₀₀ 0.2 were transferred to seven individual sterile 96-well plates. Cells were centrifuged (3000 g for 5 min), harvested, washed with 100 uL sterile H₂O, and resuspended in 100 uL SCD Screening Medium pH 7.0 with varying ratios of galactose/glucose. 80 uL of resuspended cells were then transferred to a sterile black 384-well clear-bottom plate, centrifuged (3000 g for 1 minute), shaken (2000 rpm for 30 seconds), and placed in a microplate reader pre-warmed to 30° C. and programmed to collect A₆₀₀ and fluorescence (gain 1200) measurements every 1 h for 15 h.

### AGTR1 Modulation Via Intracellular Nanobodies

Testing modulation of AGTR1 using intracellular nanobodies was performed, using a yeast strain harboring the humanized Gαi protein (DI DCyFIR P1 I) that was co-transformed with pYEplac195 AGTR1 and one of the following pYEplac181M2 constructs: pYEplac181M2_αPreProₙₐₜ-AGT-II (AGT-II (Gal induced)), pYEplac181M2 AT110_αPreProₙₐₜ-AGT-II (AGT-II+Nb AT110), pYEplac181M2 AT110i103_αPreProₙₐₜ-AGT-II (AGT-II+Nb AT1101103), pYEplac181M2 AT110i1_αPreProₙₐₜ-AGT-II (AGT-II+Nb AT110i1), or empty pYEplac181M2 (negative control). Four transformant yeast colonies per transformation were prepared, experiments performed, and data collected as described above in CXCR4 modulation via Periplasmic Display of Nanobody CA4139.

### Collecting and Processing Chains in the Protein Data Bank (PDB)

The sequences of all protein and nucleic acid chains deposited in the PDB (Berman et al., 2003, Nat Struct Biol 10: 980; Berman et al., 2000, Nucl Acids Res 28: 235-242) were downloaded from the PDB on 10/6/2022. Using a custom Python script, protein-only sequence entries were parsed into a new FASTA file that served as input for the program BLAST+ (Camacho et al., 2009, BMC Bioinformatics 10: 421) to create a local BLAST library of all protein chains in the PDB (738,016 total chains).

Collecting and Processing 1,336 Nanobody Structural Chains from the PDB

PDB entries containing nanobodies were identified using a 2-step procedure. In step one, an initial set of nanobody structures in the PDB was identified. In step two, the sequences of these structures to exhaustively collect all nanobody chains in the PDB using sequence similarity.

### Step One: Identifying an Initial Set of Nanobody Chains.

A web-based PDB query using the search term “nanobody” and source organism names Lama glama, Camelus dromedarius, Camelidae mixed library, Camelidae, Camelus bactrianus, and synthetic constructs returned 550 results that were downloaded in a custom table containing the PDB code, sequence, and sequence length of each PDB entry.

### Step Two: Collecting a Complete Set of Nanobody Chains Via Sequence Similarity.

Using BLAST+, the seed nanobody library file created in step one, and custom Python scripts, all sequences in the PDB that matched at least one member of the seed nanobody library using cutoffs of >50% sequence similarity, >100 aligned residues, and E-value <le-30 were identified. Using the SIFTS taxonomy resource, the matching sequences were filtered for nanobody chains using the taxonomic ids: 9844, Lama glama; 9838, Camelus dromedarius; 1579311, Camelidae mixed library; 9835, Camelidae; 9837, Camelus bactrianus; and 32630 synthetic constructs. Using a pipeline of custom Python scripts, the individual nanobody chain(s) in each PDB file were parsed, retained chains with <150 residues, and aligned the resultant set of 1,336 nanobody chains to a reference nanobody structure (PDB code 3P0G chain B) using the TMalign algorithm (Zhang et al., 2005, Nucl Acids Res 33: 2302-2309).

### Using Structural Informatics to Parse the Three Major Subclasses of Nanobody Structures

The three major subclasses of nanobody structures can be classified by the size and relative position of their CDR3 loops. Nanobody subclasses were numbered in order of their prevalence in the PDB. Nanobodies in subclass 1 typically have a longer CDR3 loop (>8 residues) that folds against or adjacent to β-strands 3, 4, 8, and 9, often adopting some degree α-helical substructure. In contrast, nanobodies in subclasses 2 and 3 form upright CDR3 loops that can be shorter (subclass 2) or longer (subclass 3). As such, nanobody subclasses 2 and 3 can be differentiated from subclass 1 by the lack residues adjacent to β-strands 3, 4, 8, and 9. The 1,336 nanobody chains in a 3-step process as described below, were classified.

### Step One: Selecting Structural Waypoints for the Different Nanobody Subclasses

Using structures of each nanobody subclass that was visually confirmed in PyMOL (Schrodinger, 2002, The PyMOL Molecular Graphics System, Version 2.4.2.), Cα atom waypoints for each of the three CDR3 subtypes were selected. Waypoints used for subclass 1 were: 1F2X, chain L, Cα-1105; 6WAQ, chain C, Cα-419; 1IEH, chain A, Cα-103; 1JTP, chain B, Cα-107; 2WZP, chain E, Cα-107. Waypoints used for subclass 2 were: 3POG, chain B, Cα-101; 3POG, chain B, Cα-102; 3POG, chain B, Cα-103; 5HM1, chain C, Cα-372; 3EZJ, chain H, Cα-103. Waypoints used for subclass 3 were: 3KIK, chain D, Cα-658; 1G9E, chain A, Cα-100.

### Step Two: Defining the Location, Volume, and Surface of Each CDR3 Classification Voxel.

Using a custom Python script and set of waypoint atoms, each terminal side chain (TSC) atom that was within 5 Å of any waypoint was collected. Using consensus network analysis (CNA) (see, Isom et al., 2015, Proc Natl Acad Sci USA 12: 5702-5707; Isom et al., 2016, Biochemistry 55: 534-542; Rowe et al., 2021, J Biol Chem 296: 100167), the set of 4,068 TSCs was triangulated and reduced to three clusters of 2,125, 1,737, and 31 TSCs. The CNA parameters used were minimum cluster size of 5, minimum split cluster size of 50, vacuum network size limit of 500, vacuum network distance limit of 4 Å, and split clusters selected. Using pHinder (Id.), cluster surfaces was calculated, establishing CDR3 classification voxels for the three nanobody subclasses.

### Step Three: Using CDR3 Classification Voxels to Collect the Nanobody Subclasses.

The TSCs of the 1,336 were evaluated using a custom python script nanobody chains against each CDR3 voxel. A subset of TSCs residing within CDR3 voxel were identified for each nanobody. These TSC location hits were used to discern the subclass of each nanobody chain. TSC location hits for subclass 3 were conclusive and distinct from subclasses 1 and 2. While most TSC location hits for subclasses 1 and 2 were definitive, two scenarios caused the heuristic to be further defined. In scenario one, a nanobody had <3 TSC locations hits that were typically located at the interface of the subclass 1 and 2 voxels. Using two addition Cα atom waypoints, nanobodies were assigned as belonging to subclass 1 or 2 (using waypoint 5JMO, chain D, Cα-100), or subclass 2 or 3 (using waypoint 7ME7, chain A, Cα-102). In scenario two, a nanobody had several TSC location hits in both subclass 1 and 2 voxels. By default, these nanobodies were assigned to subclass 1 unless their CDR3s had <9 TSC hits and the number of TSC hits in the subclass 2 voxel was more than double the number of TSC hits in the subclass 1 voxel. All 1,336 nanobody chains were definitively classified. Amino acid sequences for the 988 subclass 1, 301 subclass 2, and 47 subclass 3 nanobodies were saved in three FASTA-formatted libraries and animated GIF files depicting the TSC hits and classification voxels for each nanobody subclass were generated for visual confirmation.

### Selecting Scaffolds for Building Synthetic Nanobody Gene Libraries

Synthetic nanobody libraries were built by selecting particularly advantageous consensus scaffolds from nanobody subclasses 1 and 2. The protein sequences of subclasses 1 and 2 using SnapGene (San Diego, CA) were aligned and the results in FASTA format. The FASTA files and custom Python scripts were used to calculate the amino acid consensus at each residue position to build Python regular expressions of consensus amino acid sequences flanking CDRs 1, 2, and 3: subclass 1 (consensus before CDR1 “L.LSC..S” (SEQ ID NO:37) and after CDR1 “ . . . W.RQA” (SEQ ID NO:38)), (consensus before CDR2 “RFT.S.” (SEQ ID NO:39) and after CDR2 “ . . . . L.M..L”), (consensus before CDR3 “DT..Y.C..” (SEQ ID NO:40) and after CDR3 “..WG.G”); subclass 2 (consensus before CDR1 “LSC..S” (SEQ ID NO:41) and after CDR1 “ . . . W.R..P”), (consensus before CDR2 “FTI . . . ” and after CDR2 “N..YL”), (consensus before CDR3 “DT..Y.C..” (SEQ ID NO:40) and after CDR3 “ . . . WG.G”). CDR 1, 2, and 3 sequences of each nanobody in subclasses 1 and 2 were parsed to aggregate sets of nanobody chains with identical CDR 1, 2, and 3 loops and consensus CDR3 flanking regions. One subclass 1 (7KLW chain C: 7KLW.C) and two subclass 2 (3P0G chain B and 6DO1 chain C: 3P0G.B and 6DO1.C) nanobody chains were selected as particularly advantageous consensus scaffolds for building three distinct synthetic nanobody libraries. Each consensus scaffold was selected to have CDR3 loops of differing length (9, 3P0G.B; 14, 7KLW.C; and 15, 6DO1.C) that equally subdivided and sampled the nanobody binding interface defined by subclass 1 and 2 CDR3 classification voxels.

### Building the Synthetic Nanobody Gene Libraries

Three synthetic nanobody libraries were prepared using a 3-step process. In step one, the CDR1 residue sequences matched across three nanobody scaffolds by changing the CDR1 of 7KLW.C (SISSI) (SEQ ID NO:42) and 6DO1.C (NIFDV) (SEQ ID NO:43) to match the CDR1 of 3P0G.B (SIFSI) (SEQ ID NO:44). Additionally, F41 of 7KLW.C was changed to Y41 to match the upstream CDR1 flanks across all three scaffolds. By matching CDR1 regions, the same primer pools could be used to introduce CDR1 diversity. In step two, each scaffold gene was codon optimized for yeast expression using the web-based optimization tool by IDT-DNA (Coralville, IA), ordered as a gBlock from IDT-DNA, and subcloned into a 2μ pYEplac181 yeast expression plasmid using a NEBuilder® HiFi DNA assembly kit (E2621, New England Biolabs, Ipswich, MA). In step 3, each synthetic library was manufactured and delivered as 10 μg of linear dsDNA by Twist Bioscience (South San Francisco, CA) using the nanobody scaffold plasmids described above, and the residue positions and amino acid frequencies listed in Tables 4-6. The resultant libraries had variable CDR1 and CDR3 regions and theoretical diversities of 6.9×10⁸ (3P0G.B), 1.35×10¹¹ (6DO1.C), and 2.04×10¹² (7KLW.C).

### Example 2: Design of Periplasmic Ligand Trapping Technology

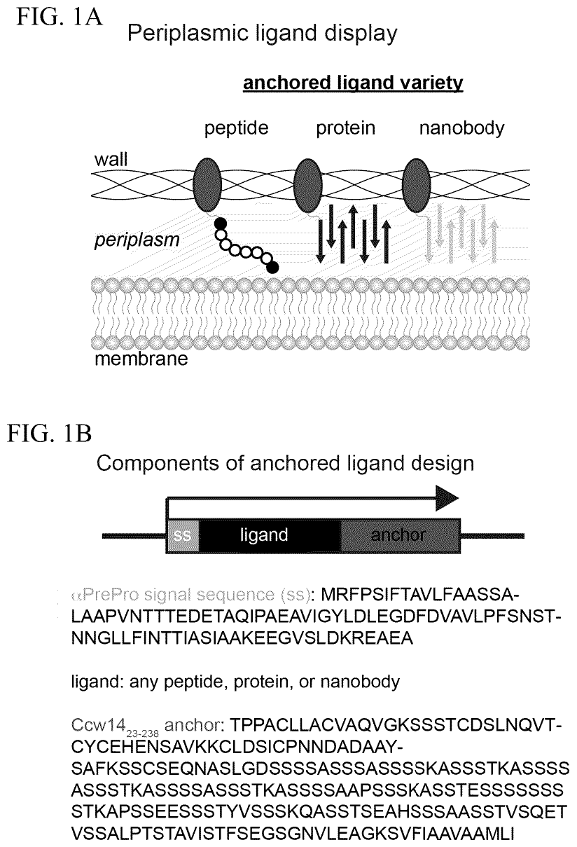

In order to design the periplasmic ligand trapping technology, identification of a suitable cell wall protein, as shown in FIG. 1A, for anchoring and displaying various types of genetically encodable ligands in the periplasmic space was performed. The purpose of identifying a suitable cell wall protein was to permit ligands to be produced by yeast, with secretion of the produced ligand, and trapping and displaying of the secreted ligand within the periplasm. Besides ligand identity itself, the design comprises two principal components: a signal sequence that promotes ligand secretion and a cell wall anchoring protein whose N terminus is projected into the periplasm. Seven different signal sequences and seven candidate anchoring proteins were selected and individually tested to identify the components required for periplasmic ligand display.

Fluorescent protein mTurqoise2 was used as a surrogate ligand to quantify total protein output, secretion efficiency, and trapping efficiency of each candidate signal sequence and anchoring protein based on fluorescence-based measurements. Results identified αPrePro (SEQ ID NO:1 and SEQ ID NO:21) as a superior signal sequence for secretion and Ccw14₂₃₋₂₃₈ (SEQ ID NO:2 and SEQ ID NO:13) as an optimal cell wall anchoring protein motif, whose amino acid sequences are shown in FIG. 1B, Table 2, and Table 3.

### Example 3: Receptor Agonism with Trapped Peptide and Protein Ligands

To confirm the ability of the yeast periplasmic trapping design for displaying ligands that activate human membrane receptors, the invention was combined with previously engineered yeast strains designed for human GPCR studies (Kapolka et al., 2020, Proc. Natl. Acad. Sci. USA 117: 13117-13126) (see Table 1). These strains contain 1) a human GPCR, 2) one of ten unique intracellular Ga proteins (humanized), and 3) a fluorescent reporter gene induced upon GPCR/Ga activation. The ability to activate Somatostatin Receptor 5 (SSTR5) through secretion and trapping of its cognate peptide ligand somatostatin (SRIF-14) was tested. As shown in FIG. 3A, periplasmic display of SRIF-14 successfully activated SSTR5, leading to activation of four different Ga proteins (G_{αi}, G_{αt}, G_{αz}, G_{α15}). These results validated the utility of the periplasmic ligand trapping technology for displaying small peptide ligands for functional modulation of a GPCR.

To confirm that larger protein ligands are also compatible with periplasmic display and functional modulation of receptors, the same design was used to display a chemokine protein ligand (CXCL12a) in strains harboring the C—X—C Motif Chemokine Receptor 4 (CXCR4). This resulted in activation of CXCR4 and two Gα proteins (G_{αi}, G_{αt}) through activated GPCR-Ga coupling, as shown in FIG. 3B. These findings authenticated the technology for displaying larger protein ligands and showcased the broad applicability of this approach for various receptor and ligand types.

TABLE 1
Intracellular Proteins

| | SEQ | | Protein | | |
|---|---|---|---|---|---|
| Protein | ID | | Sequence | | Reference |
| Name | No. | Protein Sequence (Full) | (Unique) | Strain Name | (PMID) |
| hGaᵢ | 3 | MGCTVSTQTIGDESDPFLQNKRA | ECGLY | DI DCyFIR | 32434907 |
| | | NDVIEQSLQLEKQRDKNEIKLLL | (SEQ ID | P1 I | |
| | | LGAGESGKSTVLKQLKLLHQGG | NO: 27) | | |
| | | FSHQERLQYAQVIWADAIQSMKI | | | |
| | | LIIQARKLGIQLDCDDPINNKDLF | | | |
| | | ACKRILLKAKALDYINASVAGGS | | | |
| | | DFLNDYVLKYSERYETRRRVQST | | | |
| | | GRAKAAFDEDGNISNVKSDTDR | | | |
| | | DAETVTQNEDADRNNSSRINLQD | | | |
| | | ICKDLNQEGDDQMFVRKTSREIQ | | | |
| | | GQNRRNLIHEDIAKAIKQLWNND | | | |
| | | KGIKQCFARSNEFQLEGSAAYYF | | | |
| | | DNIEKFASPNYVCTDEDILKGRIK | | | |
| | | TTGITETEFNIGSSKFKVLDAGGQ | | | |
| | | RSERKKWIHCFEGITAVLFVLAM | | | |
| | | SEYDQMLFEDERVNRMHESIML | | | |
| | | FDTLLNSKWFKDTPFILFLNKIDL | | | |
| | | FEEKVKSMPIRKYFPDYQGRVGD | | | |
| | | AEAGLKYFEKIFLSLNKTNKPIYV | | | |
| | | KRTCATDTQTMKFVLSAVTDLII | | | |
| | | QQNLKECGLY | | | |
| hGaₒ | 4 | MGCTVSTQTIGDESDPFLQNKRA | GCGLY | DI DCyFIR | 32434907 |
| | | NDVIEQSLQLEKQRDKNEIKLLL | (SEQ ID | P1 O | |
| | | LGAGESGKSTVLKQLKLLHQGG | NO: 28) | | |
| | | FSHQERLQYAQVIWADAIQSMKI | | | |
| | | LIIQARKLGIQLDCDDPINNKDLF | | | |
| | | ACKRILLKAKALDYINASVAGGS | | | |
| | | DFLNDYVLKYSERYETRRRVQST | | | |
| | | GRAKAAFDEDGNISNVKSDTDR | | | |
| | | DAETVTQNEDADRNNSSRINLQD | | | |
| | | ICKDLNQEGDDQMFVRKTSREIQ | | | |
| | | GQNRRNLIHEDIAKAIKQLWNND | | | |
| | | KGIKQCFARSNEFQLEGSAAYYF | | | |
| | | DNIEKFASPNYVCTDEDILKGRIK | | | |
| | | TTGITETEFNIGSSKFKVLDAGGQ | | | |
| | | RSERKKWIHCFEGITAVLFVLAM | | | |
| | | SEYDQMLFEDERVNRMHESIML | | | |
| | | FDTLLNSKWFKDTPFILFLNKIDL | | | |
| | | FEEKVKSMPIRKYFPDYQGRVGD | | | |
| | | AEAGLKYFEKIFLSLNKTNKPIYV | | | |
| | | KRTCATDTQTMKFVLSAVTDLII | | | |
| | | QQNLKGCGLY | | | |
| hGaₜ | 5 | MGCTVSTQTIGDESDPFLQNKRA | DCGLF | DI DCyFIR | 32434907 |
| | | NDVIEQSLQLEKQRDKNEIKLLL | (SEQ ID | P1 T | |
| | | LGAGESGKSTVLKQLKLLHQGG | NO: 29) | | |
| | | FSHQERLQYAQVIWADAIQSMKI | | | |
| | | LIIQARKLGIQLDCDDPINNKDLF | | | |
| | | ACKRILLKAKALDYINASVAGGS | | | |
| | | DFLNDYVLKYSERYETRRRVQST | | | |
| | | GRAKAAFDEDGNISNVKSDTDR | | | |
| | | DAETVTQNEDADRNNSSRINLQD | | | |
| | | ICKDLNQEGDDQMFVRKTSREIQ | | | |
| | | GQNRRNLIHEDIAKAIKQLWNND | | | |
| | | KGIKQCFARSNEFQLEGSAAYYF | | | |
| | | DNIEKFASPNYVCTDEDILKGRIK | | | |
| | | TTGITETEFNIGSSKFKVLDAGGQ | | | |
| | | RSERKKWIHCFEGITAVLFVLAM | | | |
| | | SEYDQMLFEDERVNRMHESIML | | | |
| | | FDTLLNSKWFKDTPFILFLNKIDL | | | |
| | | FEEKVKSMPIRKYFPDYQGRVGD | | | |
| | | AEAGLKYFEKIFLSLNKTNKPIYV | | | |
| | | KRTCATDTQTMKFVLSAVTDLII | | | |
| | | QQNLKDCGLF | | | |
| hGa_{z} | 6 | MGCTVSTQTIGDESDPFLQNKRA | YIGLC | DI DCyFIR | 32434907 |
| | | NDVIEQSLQLEKQRDKNEIKLLL | (SEQ ID | P1 Z | |
| | | LGAGESGKSTVLKQLKLLHQGG | NO: 30) | | |
| | | FSHQERLQYAQVIWADAIQSMKI | | | |
| | | LIIQARKLGIQLDCDDPINNKDLF | | | |
| | | ACKRILLKAKALDYINASVAGGS | | | |
| | | DFLNDYVLKYSERYETRRRVQST | | | |
| | | GRAKAAFDEDGNISNVKSDTDR | | | |
| | | DAETVTQNEDADRNNSSRINLQD | | | |
| | | ICKDLNQEGDDQMFVRKTSREIQ | | | |
| | | GQNRRNLIHEDIAKAIKQLWNND | | | |
| | | KGIKQCFARSNEFQLEGSAAYYF | | | |
| | | DNIEKFASPNYVCTDEDILKGRIK | | | |
| | | TTGITETEFNIGSSKFKVLDAGGQ | | | |
| | | RSERKKWIHCFEGITAVLFVLAM | | | |
| | | SEYDQMLFEDERVNRMHESIML | | | |
| | | FDTLLNSKWFKDTPFILFLNKIDL | | | |
| | | FEEKVKSMPIRKYFPDYQGRVGD | | | |
| | | AEAGLKYFEKIFLSLNKTNKPIYV | | | |
| | | KRTCATDTQTMKFVLSAVTDLII | | | |
| | | QQNLKYIGLC | | | |
| hGa_{q} | 7 | MGCTVSTQTIGDESDPFLQNKRA | EYNLV | DI DCyFIR | 32434907 |
| | | NDVIEQSLQLEKQRDKNEIKLLL | (SEQ ID | P1 Q | |
| | | LGAGESGKSTVLKQLKLLHQGG | NO: 31) | | |
| | | FSHQERLQYAQVIWADAIQSMKI | | | |
| | | LIIQARKLGIQLDCDDPINNKDLF | | | |
| | | ACKRILLKAKALDYINASVAGGS | | | |
| | | DFLNDYVLKYSERYETRRRVQST | | | |
| | | GRAKAAFDEDGNISNVKSDTDR | | | |
| | | DAETVTQNEDADRNNSSRINLQD | | | |
| | | ICKDLNQEGDDQMFVRKTSREIQ | | | |
| | | GQNRRNLIHEDIAKAIKQLWNND | | | |
| | | KGIKQCFARSNEFQLEGSAAYYF | | | |
| | | DNIEKFASPNYVCTDEDILKGRIK | | | |
| | | TTGITETEFNIGSSKFKVLDAGGQ | | | |
| | | RSERKKWIHCFEGITAVLFVLAM | | | |
| | | SEYDQMLFEDERVNRMHESIML | | | |
| | | FDTLLNSKWFKDTPFILFLNKIDL | | | |
| | | FEEKVKSMPIRKYFPDYQGRVGD | | | |
| | | AEAGLKYFEKIFLSLNKTNKPIYV | | | |
| | | KRTCATDTQTMKFVLSAVTDLII | | | |
| | | QQNLKEYNLV | | | |
| hGa₁₄ | 8 | MGCTVSTQTIGDESDPFLQNKRA | EFNLV | DI DCyFIR | 32434907 |
| | | NDVIEQSLQLEKQRDKNEIKLLL | (SEQ ID | P1 14 | |
| | | LGAGESGKSTVLKQLKLLHQGG | NO: 32) | | |
| | | FSHQERLQYAQVIWADAIQSMKI | | | |
| | | LIIQARKLGIQLDCDDPINNKDLF | | | |
| | | ACKRILLKAKALDYINASVAGGS | | | |
| | | DFLNDYVLKYSERYETRRRVQST | | | |
| | | GRAKAAFDEDGNISNVKSDTDR | | | |
| | | DAETVTQNEDADRNNSSRINLQD | | | |
| | | ICKDLNQEGDDQMFVRKTSREIQ | | | |
| | | GQNRRNLIHEDIAKAIKQLWNND | | | |
| | | KGIKQCFARSNEFQLEGSAAYYF | | | |
| | | DNIEKFASPNYVCTDEDILKGRIK | | | |
| | | TTGITETEFNIGSSKFKVLDAGGQ | | | |
| | | RSERKKWIHCFEGITAVLFVLAM | | | |
| | | SEYDQMLFEDERVNRMHESIML | | | |
| | | FDTLLNSKWFKDTPFILFLNKIDL | | | |
| | | FEEKVKSMPIRKYFPDYQGRVGD | | | |
| | | AEAGLKYFEKIFLSLNKTNKPIYV | | | |
| | | KRTCATDTQTMKFVLSAVTDLII | | | |
| | | QQNLKEFNLV | | | |
| hGa₁₅ | 9 | MGCTVSTQTIGDESDPFLQNKRA | EINLL | DI DCyFIR | 32434907 |
| | | NDVIEQSLQLEKQRDKNEIKLLL | (SEQ ID | P1 15 | |
| | | LGAGESGKSTVLKQLKLLHQGG | NO: 33) | | |
| | | FSHQERLQYAQVIWADAIQSMKI | | | |
| | | LIIQARKLGIQLDCDDPINNKDLF | | | |
| | | ACKRILLKAKALDYINASVAGGS | | | |
| | | DFLNDYVLKYSERYETRRRVQST | | | |
| | | GRAKAAFDEDGNISNVKSDTDR | | | |
| | | DAETVTQNEDADRNNSSRINLQD | | | |
| | | ICKDLNQEGDDQMFVRKTSREIQ | | | |
| | | GQNRRNLIHEDIAKAIKQLWNND | | | |
| | | KGIKQCFARSNEFQLEGSAAYYF | | | |
| | | DNIEKFASPNYVCTDEDILKGRIK | | | |
| | | TTGITETEFNIGSSKFKVLDAGGQ | | | |
| | | RSERKKWIHCFEGITAVLFVLAM | | | |
| | | SEYDQMLFEDERVNRMHESIML | | | |
| | | FDTLLNSKWFKDTPFILFLNKIDL | | | |
| | | FEEKVKSMPIRKYFPDYQGRVGD | | | |
| | | AEAGLKYFEKIFLSLNKTNKPIYV | | | |
| | | KRTCATDTQTMKFVLSAVTDLII | | | |
| | | QQNLKEINLL | | | |
| hGa₁₂ | 10 | MGCTVSTQTIGDESDPFLQNKRA | DIMLQ | DI DCyFIR | 32434907 |
| | | NDVIEQSLQLEKQRDKNEIKLLL | (SEQ ID | P1 12 | |
| | | LGAGESGKSTVLKQLKLLHQGG | NO: 34) | | |
| | | FSHQERLQYAQVIWADAIQSMKI | | | |
| | | LIIQARKLGIQLDCDDPINNKDLF | | | |
| | | ACKRILLKAKALDYINASVAGGS | | | |
| | | DFLNDYVLKYSERYETRRRVQST | | | |
| | | GRAKAAFDEDGNISNVKSDTDR | | | |
| | | DAETVTQNEDADRNNSSRINLQD | | | |
| | | ICKDLNQEGDDQMFVRKTSREIQ | | | |
| | | GQNRRNLIHEDIAKAIKQLWNND | | | |
| | | KGIKQCFARSNEFQLEGSAAYYF | | | |
| | | DNIEKFASPNYVCTDEDILKGRIK | | | |
| | | TTGITETEFNIGSSKFKVLDAGGQ | | | |
| | | RSERKKWIHCFEGITAVLFVLAM | | | |
| | | SEYDQMLFEDERVNRMHESIML | | | |
| | | FDTLLNSKWFKDTPFILFLNKIDL | | | |
| | | FEEKVKSMPIRKYFPDYQGRVGD | | | |
| | | AEAGLKYFEKIFLSLNKTNKPIYV | | | |
| | | KRTCATDTQTMKFVLSAVTDLII | | | |
| | | QQNLKDIMLQ | | | |
| hGa₁₃ | 11 | MGCTVSTQTIGDESDPFLQNKRA | QLMLQ | DI DCyFIR | 32434907 |
| | | NDVIEQSLQLEKQRDKNEIKLLL | (SEQ ID | P1 13 | |
| | | LGAGESGKSTVLKQLKLLHQGG | NO: 35) | | |
| | | FSHQERLQYAQVIWADAIQSMKI | | | |
| | | LIIQARKLGIQLDCDDPINNKDLF | | | |
| | | ACKRILLKAKALDYINASVAGGS | | | |
| | | DFLNDYVLKYSERYETRRRVQST | | | |
| | | GRAKAAFDEDGNISNVKSDTDR | | | |
| | | DAETVTQNEDADRNNSSRINLQD | | | |
| | | ICKDLNQEGDDQMFVRKTSREIQ | | | |
| | | GQNRRNLIHEDIAKAIKQLWNND | | | |
| | | KGIKQCFARSNEFQLEGSAAYYF | | | |
| | | DNIEKFASPNYVCTDEDILKGRIK | | | |
| | | TTGITETEFNIGSSKFKVLDAGGQ | | | |
| | | RSERKKWIHCFEGITAVLFVLAM | | | |
| | | SEYDQMLFEDERVNRMHESIML | | | |
| | | FDTLLNSKWFKDTPFILFLNKIDL | | | |
| | | FEEKVKSMPIRKYFPDYQGRVGD | | | |
| | | AEAGLKYFEKIFLSLNKTNKPIYV | | | |
| | | KRTCATDTQTMKFVLSAVTDLII | | | |
| | | QQNLKQLMLQ | | | |
| hGaₛ | 12 | MGCTVSTQTIGDESDPFLQNKRA | QYELL | DI DCyFIR | 32434907 |
| | | NDVIEQSLQLEKQRDKNEIKLLL | (SEQ ID | P1 S | |
| | | LGAGESGKSTVLKQLKLLHQGG | NO: 36) | | |
| | | FSHQERLQYAQVIWADAIQSMKI | | | |
| | | LIIQARKLGIQLDCDDPINNKDLF | | | |
| | | ACKRILLKAKALDYINASVAGGS | | | |
| | | DFLNDYVLKYSERYETRRRVQST | | | |
| | | GRAKAAFDEDGNISNVKSDTDR | | | |
| | | DAETVTQNEDADRNNSSRINLQD | | | |
| | | ICKDLNQEGDDQMFVRKTSREIQ | | | |
| | | GQNRRNLIHEDIAKAIKQLWNND | | | |
| | | KGIKQCFARSNEFQLEGSAAYYF | | | |
| | | DNIEKFASPNYVCTDEDILKGRIK | | | |
| | | TTGITETEFNIGSSKFKVLDAGGQ | | | |
| | | RSERKKWIHCFEGITAVLFVLAM | | | |
| | | SEYDQMLFEDERVNRMHESIML | | | |
| | | FDTLLNSKWFKDTPFILFLNKIDL | | | |
| | | FEEKVKSMPIRKYFPDYQGRVGD | | | |
| | | AEAGLKYFEKIFLSLNKTNKPIYV | | | |
| | | KRTCATDTQTMKFVLSAVTDLII | | | |
| | | QQNLKQYELL | | | |

TABLE 2
Anchor Proteins

| | SEQ ID | | Alternative | Reference |
|---|---|---|---|---|
| Name | No. | Protein Sequence | Name | (PMID) |
| Ccw14₂₃₋₂₃₈ | 13* | TPPACLLACVAQVGKSSST | — | — |
| | | CDSLNQVTCYCEHENSAV | | |
| | | KKCLDSICPNNDADAAYS | | |
| | | AFKSSCSEQNASLGDSSSS | | |
| | | ASSSASSSSKASSSTKASSS | | |
| | | SASSSTKASSSSASSSTKAS | | |
| | | SSSAAPSSSKASSTESSSSSS | | |
| | | SSTKAPSSEESSSTYVSSSK | | |
| | | QASSTSEAHSSSAASSTVS | | |
| | | QETVSSALPTSTAVISTFSE | | |
| | | GSGNVLEAGKSVFIAAVA | | |
| | | AMLI | | |
| Sed1₁₉₋₃₃₈ | 14** | QFSNSTSASSTDVTSSSSIST | — | — |
| | | SSGSVTITSSEAPESDNGTS | | |
| | | TAAPTETSTEAPTTAIPING | | |
| | | TSTEAPTTAIPTNGTSTEAP | | |
| | | TDTTTEAPTTALPTNGTST | | |
| | | EAPTDTTTEAPTTGLPTNG | | |
| | | TTSAFPPTTSLPPSNTTTTPP | | |
| | | YNPSTDYTTDYTVVTEYTT | | |
| | | YCPEPTTFTTNGKTYTVTE | | |
| | | PTTLTITDCPCTIEKPTTTST | | |
| | | TEYTVVTEYTTYCPEPTTF | | |
| | | TTNGKTYTVTEPTTLTITD | | |
| | | CPCTIEKSEAPESSVPVTES | | |
| | | KGTTTKETGVTTKQTTANP | | |
| | | SLTVSTVVPVSSSASSHSV | | |
| | | VINSNGANVVVPGALGLA | | |
| | | GVAMLFL | | |
| Flo11₄₉₆₋₁₅₃₇ | 15** | ASSMVGYSTASLEISTYAG | Flo42 | 22623985, |
| | | SANSLLAGSGLSVFIASLLL | | 32358068 |
| | | AII | | |
| Flo5₂₅₋₁₀₇₅ | 16 | ATEACLPAGQRKSGMNINF | — | — |
| | | YQYSLKDSSTYSNAAYMA | | |
| | | YGYASKTKLGSVGGQTDIS | | |
| | | IDYNIPCVSSSGTFPCPQED | | |
| | | SYGNWGCKGMGACSNSQ | | |
| | | GIAYWSTDLFGFYTTPTNV | | |
| | | TLEMTGYFLPPQTGSYTFS | | |
| | | FATVDDSAILSVGGSIAFEC | | |
| | | CAQEQPPITSTNFTINGIKP | | |
| | | WDGSLPDNITGTVYMYAG | | |
| | | YYYPLKVVYSNAVSWGTL | | |
| | | PISVELPDGTTVSDNFEGY | | |
| | | VYSFDDDLSQSNCTIPDPSI | | |
| | | HTTSTITTTTEPWTGTFTST | | |
| | | STEMTTITDTNGQLTDETVI | | |
| | | VIRTPTTASTITTTTEPWTG | | |
| | | TFTSTSTEMTTVTGTNGQP | | |
| | | TDETVIVIRTPTSEGLITTTT | | |
| | | EPWTGTFTSTSTEMTTVTG | | |
| | | TNGQPTDETVIVIRTPTSEG | | |
| | | LITTTTEPWTGTFTSTSTEV | | |
| | | TTITGTNGQPTDETVIVIRT | | |
| | | PTSEGLITTTTEPWTGTFTS | | |
| | | TSTEMTTVTGTNGQPTDET | | |
| | | VIVIRTPTSEGLISTTTEPWT | | |
| | | GTFTSTSTEVTTITGTNGQP | | |
| | | TDETVIVIRTPTSEGLITTTT | | |
| | | EPWTGTFTSTSTEMTTVTG | | |
| | | TNGQPTDETVIVIRTPTSEG | | |
| | | LITRTTEPWTGTFTSTSTEV | | |
| | | TTITGTNGQPTDETVIVIRT | | |
| | | PTTAISSSLSSSSGQITSSITS | | |
| | | SRPIITPFYPSNGTSVISSSVI | | |
| | | SSSVTSSLVTSSSFISSSVISS | | |
| | | STTTSTSIFSESSTSSVIPTSS | | |
| | | STSGSSESKTSSASSSSSSSS | | |
| | | ISSESPKSPTNSSSSLPPVTS | | |
| | | ATTGQETASSLPPATTTKT | | |
| | | SEQTTLVTVTSCESHVCTE | | |
| | | SISSAIVSTATVTVSGVTTE | | |
| | | YTTWCPISTTETTKQTKGT | | |
| | | TEQTKGTTEQTTETTKQTT | | |
| | | VVTISSCESDICSKTASPAIV | | |
| | | STSTATINGVTTEYTTWCPI | | |
| | | STTESKQQTTLVTVTSCES | | |
| | | GVCSETTSPAIVSTATATV | | |
| | | NDVVTVYPTWRPQTTNEQ | | |
| | | SVSSKMNSATSETTTNTGA | | |
| | | AETKTAVTSSLSRFNHAET | | |
| | | QTASATDVIGHSSSVVSVS | | |
| | | ETGNTMSLTSSGLSTMSQQ | | |
| | | PRSTPASSMVGSSTASLEIS | | |
| | | TYAGSANSLLAGSGLSVFI | | |
| | | ASLLLAII | | |
| Suc2₂₀₋₅₃₂ | 17** | SMTNETSDRPLVHFTPNKG | — | — |
| | | WMNDPNGLWYDEKDAK | | |
| | | WHLYFQYNPNDTVWGTPL | | |
| | | FWGHATSDDLTNWEDQPI | | |
| | | AIAPKRNDSGAFSGSMVV | | |
| | | DYNNTSGFFNDTIDPRQRC | | |
| | | VAIWTYNTPESEEQYISYSL | | |
| | | DGGYTFTEYQKNPVLAAN | | |
| | | STQFRDPKVFWYEPSQKWI | | |
| | | MTAAKSQDYKIEIYSSDDL | | |
| | | KSWKLESAFANEGFLGYQ | | |
| | | YECPGLIEVPTEQDPSKSY | | |
| | | WVMFISINPGAPAGGSFNQ | | |
| | | YFVGSFNGTHFEAFDNQSR | | |
| | | VVDFGKDYYALQTFFNTD | | |
| | | PTYGSALGIAWASNWEYS | | |
| | | AFVPTNPWRSSMSLVRKFS | | |
| | | LNTEYQANPETELINLKAE | | |
| | | PILNISNAGPWSRFATNTTL | | |
| | | TKANSYNVDLSNSTGTLEF | | |
| | | ELVYAVNTTQTISKSVFAD | | |
| | | LSLWFKGLEDPEEYLRMG | | |
| | | FEVSASSFFLDRGNSKVKF | | |
| | | VKENPYFTNRMSVNNQPF | | |
| | | KSENDLSYYKVYGLLDQNI | | |
| | | LELYFNDGDVVSTNTYFM | | |
| | | TTGNALGSVNMTTGVDNL | | |
| | | FYIDKFQVREVK | | |
| Ecm3₃₂₀₋₄₂₉ | 18 | ANSTTSIPSSCSIGTSATAT | — | — |
| | | AQADLDKISGCSTIVGNLTI | | |
| | | TGDLGSAALASIQEIDGSLT | | |
| | | IFNSSSLSSFSADSIKKITGD | | |
| | | LNMQELIILTSASFGSLQEV | | |
| | | DSINMVTLPAISTESTDLQN | | |
| | | ANNIIVSDTTLESVEGFSTL | | |
| | | KKVNVFNINNNRYLNSFQS | | |
| | | SLESVSDSLQFSSNGDNTT | | |
| | | LAFDNLVWANNITLRDVN | | |
| | | SISFGSLQTVNASLGFINNT | | |
| | | LPSLNLTQLSKVGQSLSIVS | | |
| | | NDELSKAAFSNLTTVGGGF | | |
| | | IIANNTQLKVIDGFNKVQT | | |
| | | VGGAIEVTGNFSTLDLSSL | | |
| | | KSVRGGANFDSSSSNFSCN | | |
| | | ALKKLQSNGAIQGDSFVCK | | |
| | | NGATSTSVKLSSTSTESSKS | | |
| | | SATSSASSSGDASNAQANV | | |
| | | SASASSSSSSSKKSKGAAPE | | |
| | | LVPATSFMGVVAAVGVAL | | |
| | | L | | |
| Yps1₂₂₋₅₆₉ | 19 | KIIPAANKRDDDSNSKFVK | — | — |
| | | LPFHKLYGDSLENVGSDK | | |
| | | KPEVRLLKRADGYEEIIITN | | |
| | | QQSFYSVDLEVGTPPQNVT | | |
| | | VLVDTGSSDLWIMGSDNP | | |
| | | YCSSNSMGSSRRRVIDKRD | | |
| | | DSSSGGSLINDINPFGWLTG | | |
| | | TGSAIGPTATGLGGGSGTA | | |
| | | TQSVPASEATMDCQQYGT | | |
| | | FSTSGSSTFRSNNTYFSISY | | |
| | | GDGTFASGTFGTDVLDLSD | | |
| | | LNVTGLSFAVANETNSTM | | |
| | | GVLGIGLPELEVTYSGSTA | | |
| | | SHSGKAYKYDNFPIVLKNS | | |
| | | GAIKSNTYSLYLNDSDAM | | |
| | | HGTILFGAVDHSKYTGTLY | | |
| | | TIPIVNTLSASGFSSPIQFDV | | |
| | | TINGIGISDSGSSNKTLTTT | | |
| | | KIPALLDSGTTLTYLPQTV | | |
| | | VSMIATELGAQYSSRIGYY | | |
| | | VLDCPSDDSMEIVFDFGGF | | |
| | | HINAPLSSFILSTGTTCLLGI | | |
| | | IPTSDDTGTILGDSFLTNAY | | |
| | | VVYDLENLEISMAQARYN | | |
| | | TTSENIEIITSSVPSAVKAPG | | |
| | | YTNTWSTSASIVTGGNIFT | | |
| | | VNSSQTASFSGNLTTSTAS | | |
| | | ATSTSSKRNVGDHIVPSLPL | | |
| | | TLISLLFAFI | | |

*denotes best anchor protein sequence
**denotes suitable alternative

TABLE 3
Signal Sequences

| | SEQ ID | | | Reference |
|---|---|---|---|---|
| Name | No. | Protein Sequence | Alternative Name | (PMID) |
| αPreₙₐₜ | 20 | MRFPSIFTAVLFAASSALA | — | — |
| αPreProₙₐₜ | 21* | MRFPSIFTAVLFAASSALAAP | — | — |
| | | VNTTTEDETAQIPAEAVIGYL | | |
| | | DLEGDFDVAVLPFSNSTNNG | | |
| | | LLFINTTIASIAAKEEGVSLDK | | |
| | | REAEA | | |
| αPreProᵥ₂ | 22 | MRFPSIFTDVLFAASSALATP | a_{A9D,A20T}, a_{OPT} | 33687500 |
| | | VNTTTEDETAQIPAEAVIGYS | | |
| | | DLEGDFDVAVLPFSNSTNNG | | |
| | | LLFINTTIASIAAKEEGVSLEK | | |
| | | REAEAEF | | |
| αPreProᵥ₃ | 23 | MRFPSIFTDVLFAASSALATP | a_{A9D,A20T,A87T} | 33687500 |
| | | VNTTTEDETAQIPAEAVIGYS | | |
| | | DLEGDFDVAVLPFSNSTNNG | | |
| | | LLFINTTIASIAAKEEGVSLEK | | |
| | | RETEAEF | | |
| αPreProᵥ₄ | 24** | MRFPSIFTAVLFAASSALAAP | app8 | 19459139 |
| | | ANTTTEDETAQIPAEAVIDYS | | |
| | | DLEGDFDAAALPLSNSTNNG | | |
| | | LSSTNTTIASIAAKEEGVSLDK | | |
| | | REAEA | | |
| Ost1 | 25 | MRQVWFSWIVGFLCFFNVSS | — | — |
| | | A | | |
| Ost1αProₙₐₜ | 26** | MRQVWFSWIVGFLCFFNVSS | Ost1-pro-af, | 25164324, |
| | | AAPVNTTTEDETAQIPAEAVI | Ost1ss-aPro | 35610225 |
| | | GYLDLEGDFDVAVLPFSNST | | |
| | | NNGLLFINTTIASIAAKEEGVS | | |
| | | LDKREAEA | | |

*denotes best signal sequence
**denotes suitable alternative

TABLE 4
3P0G.B Library

| Variant | 1 | 2 | 3 | 4 | 5 | 6 | 7 | 8 |
|---|---|---|---|---|---|---|---|---|
| ORF-AA Position | 30 | 31 | 32 | 101 | 104 | 105 | 106 | 107 |
| ORF AA | F | S | I | Y | V | L | Y | E |
| ORF- Codon | TTC | AGC | ATT | TAT | GTC | CTG | TAT | GAA |
| A | | | | | | | | |
| C | 0.025 | 0.1 | 0.1 | 0.05 | 0.05 | 0.05 | 0.05 | 0.05 |
| D | 0.025 | 0.05 | 0.05 | 0.05 | 0.05 | 0.05 | 0.05 | 0.05 |
| E | 0.025 | 0.05 | 0.05 | 0.05 | 0.05 | 0.05 | 0.05 | 0.15 |
| F | 0.5 | 0.1 | 0.1 | 0.05 | 0.05 | 0.05 | 0.05 | 0.05 |
| G | | | | | | | | |
| H | 0.1 | 0.1 | 0.1 | 0.1 | 0.1 | 0.1 | 0.1 | 0.1 |
| I | | | | | | | | |
| K | 0.05 | 0.05 | 0.05 | 0.05 | 0.05 | 0.05 | 0.05 | 0.05 |
| L | 0.05 | 0.1 | 0.1 | 0.1 | 0.1 | 0.2 | 0.1 | 0.1 |
| M | | 0.05 | 0.05 | 0.05 | 0.05 | 0.05 | 0.05 | 0.05 |
| N | 0.05 | 0.1 | 0.1 | 0.1 | 0.1 | 0.1 | 0.1 | 0.1 |
| P | | | | | | | | |
| Q | 0.05 | 0.1 | 0.1 | 0.1 | 0.1 | 0.1 | 0.1 | 0.1 |
| R | 0.025 | 0.05 | 0.05 | 0.05 | 0.05 | 0.05 | 0.05 | 0.05 |
| S | | | | | | | | |
| T | | | | | | | | |
| V | | 0.05 | 0.05 | 0.05 | 0.15 | 0.05 | 0.05 | 0.05 |
| W | | | | | | | | |
| Y | 0.1 | 0.1 | 0.1 | 0.2 | 0.1 | 0.1 | 0.2 | 0.1 |
| Variant AA's | 11 | 13 | 13 | 13 | 13 | 13 | 13 | 13 |
| wt % | | | | | | | | |
| Other AA % | | | | | | | | |
| Total % | 1 | 1 | 1 | 1 | 1 | 1 | 1 | 1 |

TABLE 5
6DO1.C Library

| Variant | 1 | 2 | 3 | 4 | 5 | 6 | 7 | 8 | 9 | 10 |
|---|---|---|---|---|---|---|---|---|---|---|
| ORF-AA Position | 30 | 31 | 32 | 104 | 106 | 107 | 108 | 110 | 111 | 113 |
| ORF AA | F | S | I | I | T | Y | F | Y | D | D |
| ORF- Codon | TTC | AGC | ATT | ATC | ACG | TAC | TTC | TAT | GAC | GAC |
| A | | | | | | | | | | |
| C | 0.025 | 0.1 | 0.1 | 0.05 | 0.05 | 0.05 | 0.05 | 0.05 | 0.05 | 0.05 |
| D | 0.025 | 0.05 | 0.05 | 0.05 | 0.05 | 0.05 | 0.05 | 0.05 | 0.15 | 0.15 |
| E | 0.025 | 0.05 | 0.05 | 0.05 | 0.05 | 0.05 | 0.05 | 0.05 | 0.05 | 0.05 |
| F | 0.5 | 0.1 | 0.1 | 0.05 | 0.05 | 0.05 | 0.15 | 0.05 | 0.05 | 0.05 |
| G | | | | | | | | | | |
| H | 0.1 | 0.1 | 0.1 | 0.1 | 0.1 | 0.1 | 0.1 | 0.1 | 0.1 | 0.1 |
| I | | | | 0.1 | | | | | | |
| K | 0.05 | 0.05 | 0.05 | 0.05 | 0.05 | 0.05 | 0.05 | 0.05 | 0.05 | 0.05 |
| L | 0.05 | 0.1 | 0.1 | 0.1 | 0.1 | 0.1 | 0.1 | 0.1 | 0.1 | 0.1 |
| M | | 0.05 | 0.05 | 0.05 | 0.05 | 0.05 | 0.05 | 0.05 | 0.05 | 0.05 |
| N | 0.05 | 0.1 | 0.1 | 0.1 | 0.1 | 0.1 | 0.1 | 0.1 | 0.1 | 0.1 |
| P | | | | | | | | | | |
| Q | 0.05 | 0.1 | 0.1 | 0.1 | 0.1 | 0.1 | 0.1 | 0.1 | 0.1 | 0.1 |
| R | 0.025 | 0.05 | 0.05 | 0.05 | 0.05 | 0.05 | 0.05 | 0.05 | 0.05 | 0.05 |
| S | | | | | | | | | | |
| T | | | | | 0.1 | | | | | |
| V | | 0.05 | 0.05 | 0.05 | 0.05 | 0.05 | 0.05 | 0.05 | 0.05 | 0.05 |
| W | | | | | | | | | | |
| Y | 0.1 | 0.1 | 0.1 | 0.1 | 0.1 | 0.2 | 0.1 | 0.2 | 0.1 | 0.1 |
| Variant AA's | 11 | 13 | 13 | 14 | 14 | 13 | 13 | 13 | 13 | 13 |
| wt % | | | | | | | | | | |
| Other AA % | | | | | | | | | | |
| Total % | 1 | 1 | 1 | 1 | 1 | 1 | 1 | 1 | 1 | 1 |

TABLE 6
7KLW.C Library

| Variant | 1 | 2 | 3 | 4 | 5 | 6 | 7 | 8 | 9 | 10 | 11 |
|---|---|---|---|---|---|---|---|---|---|---|---|
| ORF-AA Position | 33 | 34 | 35 | 105 | 107 | 108 | 109 | 110 | 112 | 114 | 115 |
| ORF AA | F | S | I | W | Y | A | W | P | H | D | D |
| ORF- Codon | TTC | AGC | ATT | TGG | TAT | GCG | TGG | CCT | CAC | GAC | GAC |
| A | | | | | | 0.1 | | | | | |
| C | 0.025 | 0.1 | 0.1 | 0.05 | 0.05 | 0.05 | 0.05 | 0.05 | 0.05 | 0.05 | 0.05 |
| D | 0.025 | 0.05 | 0.05 | 0.05 | 0.05 | 0.05 | 0.05 | 0.05 | 0.05 | 0.15 | 0.15 |
| E | 0.025 | 0.05 | 0.05 | 0.05 | 0.05 | 0.05 | 0.05 | 0.05 | 0.05 | 0.05 | 0.05 |
| F | 0.5 | 0.1 | 0.1 | 0.05 | 0.05 | 0.05 | 0.05 | 0.05 | 0.05 | 0.05 | 0.05 |
| G | | | | | | | | | | | |
| H | 0.1 | 0.1 | 0.1 | 0.1 | 0.1 | 0.1 | 0.1 | 0.1 | 0.2 | 0.1 | 0.1 |
| I | | | | | | | | | | | |
| K | 0.05 | 0.05 | 0.05 | 0.05 | 0.05 | 0.05 | 0.05 | 0.05 | 0.05 | 0.05 | 0.05 |
| L | 0.05 | 0.1 | 0.1 | 0.1 | 0.1 | 0.1 | 0.1 | 0.1 | 0.1 | 0.1 | 0.1 |
| M | | 0.05 | 0.05 | 0.05 | 0.05 | 0.05 | 0.05 | 0.05 | 0.05 | 0.05 | 0.05 |
| N | 0.05 | 0.1 | 0.1 | 0.1 | 0.1 | 0.1 | 0.1 | 0.1 | 0.1 | 0.1 | 0.1 |
| P | | | | | | | | 0.1 | | | |
| Q | 0.05 | 0.1 | 0.1 | 0.1 | 0.1 | 0.1 | 0.1 | 0.1 | 0.1 | 0.1 | 0.1 |
| R | 0.025 | 0.05 | 0.05 | 0.05 | 0.05 | 0.05 | 0.05 | 0.05 | 0.05 | 0.05 | 0.05 |
| S | | | | | | | | | | | |
| T | | | | | | | | | | | |
| V | | 0.05 | 0.05 | 0.05 | 0.05 | 0.05 | 0.05 | 0.05 | 0.05 | 0.05 | 0.05 |
| W | | | | 0.1 | | | 0.1 | | | | |
| Y | 0.1 | 0.1 | 0.1 | 0.1 | 0.2 | 0.1 | 0.1 | 0.1 | 0.1 | 0.1 | 0.1 |
| Variant AA's | 11 | 13 | 13 | 14 | 13 | 14 | 14 | 14 | 13 | 13 | 13 |
| wt % | | | | | | | | | | | |
| Other AA % | | | | | | | | | | | |
| Total % | 1 | 1 | 1 | 1 | 1 | 1 | 1 | 1 | 1 | 1 | 1 |

### Example 4: Chemokine Receptor Antagonism Via Trapped Nanobodies

The periplasmic ligand trapping applications from FIG. 3A-3B demonstrated the technology's compatibility with ligands of various sizes but only provides an example of receptor agonism. Additionally, these examples involve displaying endogenous peptide/protein ligands rather than synthetic ligands, e.g., nanobodies, designed with specified pharmacological properties. The ability to antagonize CXCR4 signaling using a synthetic nanobody (CA4139) was tested in order to validate the periplasmic ligand trapping technology can accommodate antagonistic ligands and/or non-endogenous synthetic ligands.

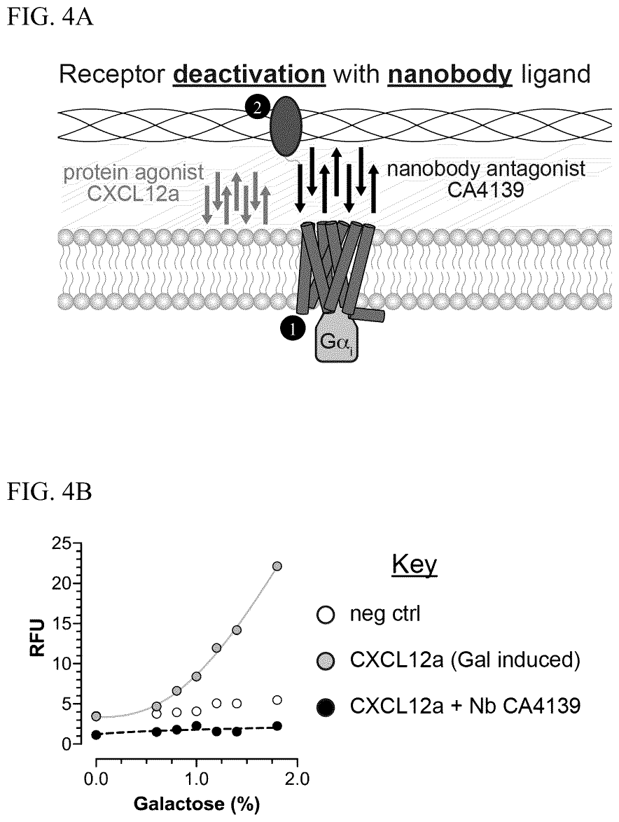

Using engineered yeast strains harboring CXCR4 as described in FIG. 3A-3B, the periplasmic ligand trapping technology to display nanobody CA4139 (Claes et al., 2016, ACS Synth Biol 5: 1070-1075) was used to examine if the CXCL12a-derived activation of CXCR4 could be antagonized. As shown in FIG. 4A-4B, this approach was successful, leading to complete loss of CXCR4 signaling in the presence of CA4139.

### Example 5: Functional Assessment of Receptor-Nanobody Intracellular Interactions

Incorporating the ligand trapping technology into the yeast platform for human membrane receptor studies allows assessment of receptor function from peptide-, protein-, and nanobody-receptor interactions occurring at/near the orthosteric binding site. However, identifying similar interactions that occur at the receptor's intracellular face can also be advantageous and used for modulating receptor function. This is especially true for nanobodies, which can recognize specific receptor conformations and influence interactions between receptors and intracellular signaling proteins that control downstream signaling pathways.

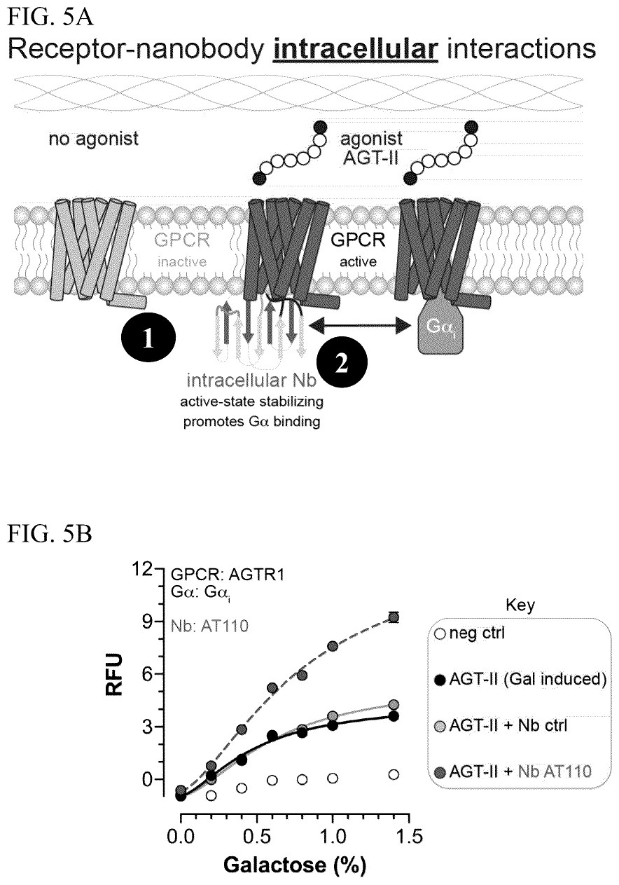

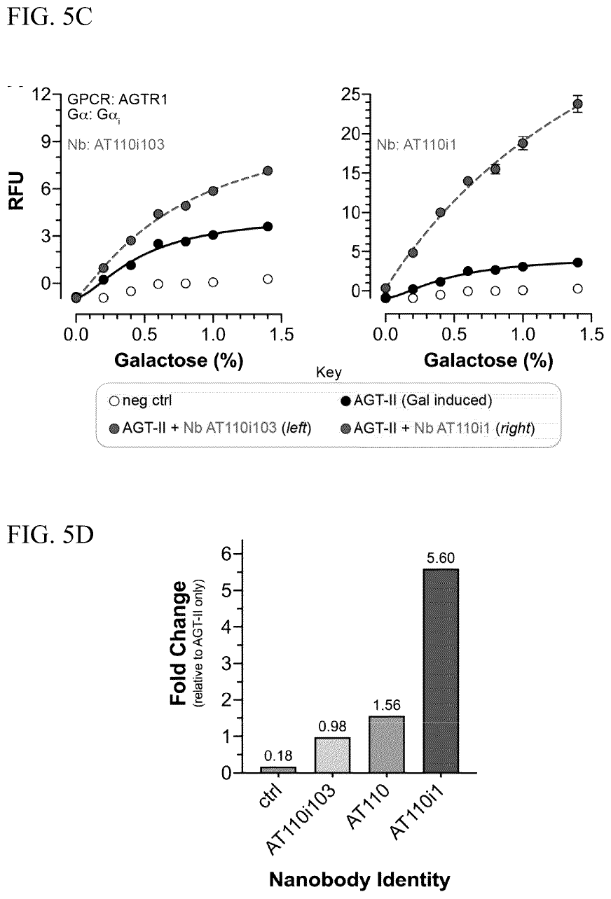

Using engineered yeast strains harboring Angiotensin II Receptor Type I (AGTR1), autocrine activation of this receptor was tested to see co-expression/secretion of its cognate peptide ligand angiotensin II (AGT-II). As shown in FIG. 5B (black), inducible expression of AGT-II successfully resulted in AGTR1 activation, providing another example of the ability to design autocrine signaling systems described previously. Co-expressed intracellular nanobody AT110 (Wingler et al., 2019, Cell 176: 479-490) to determine if functionally relevant nanobody-receptor interactions could be detected and how these interactions ultimately influence downstream signaling. As shown in FIG. 5B, AT110-AGTR1 interactions were detected and resulted in increased signaling relative to AGT-II only (i.e., no nanobody). To confirm this increased signaling was caused by AT110-AGTR1 interactions, additional experiments where AT110 was replaced with a non-specific nanobody were performed. This resulted in no change in signaling (sec FIG. 5B, gray) relative to AGT-II only, validating the increased signaling observed with AT110 was the direct result of nanobody-receptor interactions.

Initial studies identifying AT110 indicated this nanobody stabilizes the active state of AGTR1 (Wingler et al., 2019, Cell 176: 479-490), correlating with the increased signaling observed above. Next, two additional active-state stabilizing intracellular nanobodies for AGTR1, i.e., AT1101103 (Wellner et al., 2021, Nat Chem Biol 17: 1057-1064) and AT110i1 (Wingler et al., 2019, Cell 176: 479-490) were tested, to confirm the platform was capable of reporting similar nanobody-receptor interactions. As shown in FIGS. 5C-5D, both intracellular nanobodies AT1101103 and AT110i1 resulted in increased signaling as seen with AT110 but with unique levels for each nanobody. These results confirmed the platform's ability to identify intracellular nanobody-receptor interactions and its capability of precisely detecting minor differences that can result in major signaling outcomes.

### Example 6: Building and Screening Synthetic Nanobody Libraries that Regulate GPCRs

A major application of the nanobody trapping platform disclosed herein is the discovery of extracellular nanobodies that regulate GPCR function by acting as full and partial agonists, inverse agonists, positive and negative allosteric modulators, and antagonists. Additionally, this platform is particularly advantageous for discovering intracellular nanobodies that function as GPCR chaperones, are selective for ligand-dependent active states, are selective for inactive states, and can identify specific GPCR isoforms. In both cases, such nanobodies could serve as novel and essential tools for studying GPCR pharmacology, developing GPCR-targeted nanobody therapies, and providing new biotechnologies based on specific GPCR-nanobody interactions.

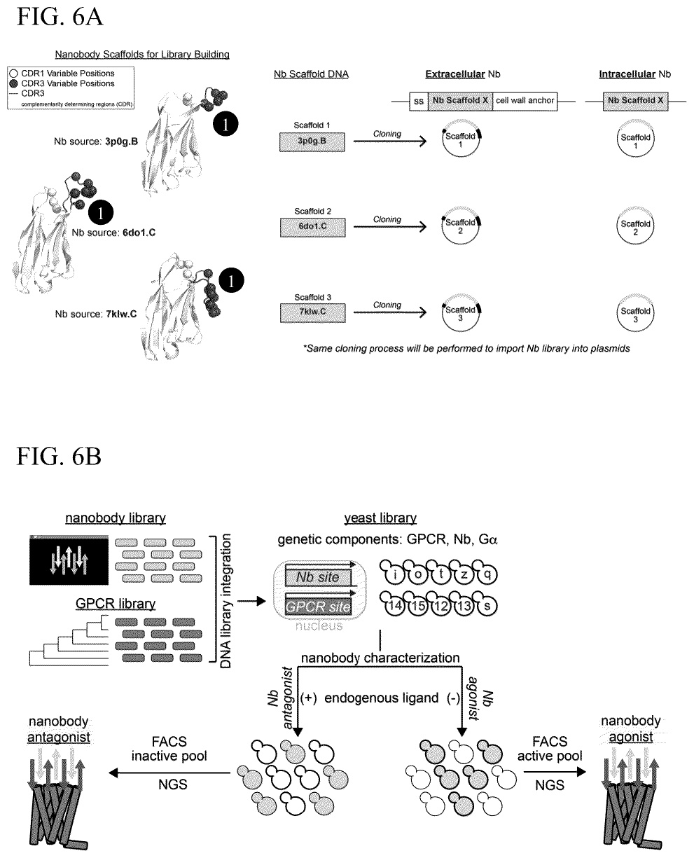

Three synthetic nanobody libraries that are compatible with the nanobody discovery platform were designed and manufactured. As detailed in the materials and methods, particularly advantageous consensus nanobody scaffolds from the successful examples available in the Protein Data Bank (PDB) (Berman et al., 2003, Nat Struct Biol 10: 980; Berman et al., 2000, Nucl Acids Res 28: 235-242) were identified. Using custom Python code 1,336 nanobody chains from the PDB into subclasses 1 [988 (74%) chains], 2, [301 (23%) chains], and 3 [47 (3%) chains] based on the position and conformation of their third complementarity determining region (CDR3) loops (FIG. 6A) were classified and identified. Performing sequence and structure-based calculations on subclasses 1 and 2 nanobodies, a subset of particularly advantageous consensus scaffolds was identified. From this subset, one subclass 1 (7KLW chain C: 7KLW.C) and two subclass 2 (3P0G chain B and 6DO1 chain C: 3P0G.B and 6DO1.C) nanobody chains as particularly advantageous consensus scaffolds were selected. Using these scaffolds, amino acid diversity into CDR1 and CDR3 was introduced to created three distinct synthetic nanobody libraries with theoretical diversities of 6.9×10⁸ (3P0G.B), 1.35×10¹¹ (6DO1.C), and 2.04×10¹² (7KLW.C).

Each library can be screened for new nanobody-GPCR interactions by PCR amplifying and transforming a pool of nanobody amplicons into DCyFIR yeast strains (Kapolka et al., 2020, Proc. Natl. Acad. Sci. USA 117: 13117-13126; Rowe et al., 2020, J. Biol. Chem. 295: 8262-8271). Using this approach, nanobody genes can either be integrated into expression vectors or integrated directly into the genome via CRISPR (FIG. 6B). The resultant libraries can then be screened and validated using fluorescence activated cell sorting (Kapolka et al., 2020, Proc. Natl. Acad. Sci. USA 117: 13117-13126; Rowe et al., 2020, J. Biol. Chem. 295: 8262-8271) and a variety of readouts, including Sanger sequencing, quantitative PCR, or next generation sequencing (FIG. 6B).

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

### REFERENCES

1. J. Goedhart et al., Structure-guided evolution of cyan fluorescent proteins towards a quantum yield of 93%. Nat Commun 3, 751 (2012).
2. N. J. Kapolka et al., DCyFIR: a high-throughput CRISPR platform for multiplexed G protein-coupled receptor profiling and ligand discovery. Proc Natl Acad Sci USA 117, 13117-13126 (2020).
3. J. B. Rowe, G. J. Taghon, N. J. Kapolka, W. M. Morgan, D. G. Isom, CRISPR-addressable yeast strains with applications in human G protein-coupled receptor profiling and synthetic biology. J Biol Chem 295, 8262-8271 (2020).
4. K. Claes et al., Modular Integrated Secretory System Engineering in Pichia pastoris To Enhance G-Protein Coupled Receptor Expression. ACS Synth Biol 5, 1070-1075 (2016).
5. L. M. Wingler, C. McMahon, D. P. Staus, R. J. Lefkowitz, A. C. Kruse, Distinctive Activation Mechanism for Angiotensin Receptor Revealed by a Synthetic Nanobody. Cell 176, 479-490 e412 (2019).
6. A. Wellner et al., Rapid generation of potent antibodies by autonomous hypermutation in yeast. Nat Chem Biol 17, 1057-1064 (2021).
7. H. Berman, K. Henrick, H. Nakamura, Announcing the worldwide Protein Data Bank. Nat Struct Biol 10, 980 (2003).
8. H. M. Berman et al., The Protein Data Bank. Nucleic Acids Res 28, 235-242 (2000).
9. J. B. Rowe, N. J. Kapolka, G. J. Taghon, W. M. Morgan, D. G. Isom, The evolution and mechanism of GPCR proton sensing. Journal of Biological Chemistry 296 (2021).
10. P. Aza, G. Molpeceres, F. de Salas, S. Camarero, Design of an improved universal signal peptide based on the alpha-factor mating secretion signal for enzyme production in yeast. Cell Mol Life Sci 78, 3691-3707 (2021).
11. J. A. Rakestraw, S. L. Sazinsky, A. Piatesi, E. Antipov, K. D. Wittrup, Directed evolution of a secretory leader for the improved expression of heterologous proteins and full-length antibodies in Saccharomyces cerevisiae. Biotechnol Bioeng 103, 1192-1201 (2009).
12. B. D. M. Bean et al., Functional expression of opioid receptors and other human GPCRs in yeast engineered to produce human sterols. Nat Commun 13, 2882 (2022).
13. I. Fitzgerald, B. S. Glick, Secretion of a foreign protein from budding yeasts is enhanced by cotranslational translocation and by suppression of vacuolar targeting. Microbial Cell Factories 13 (2014).
14. W. K. Kroeze et al., PRESTO-Tango as an open-source resource for interrogation of the druggable human GPCRome. Nat Struct Mol Biol 22, 362-369 (2015).
15. E. M. Rosenberg, Jr. et al., Characterization, Dynamics, and Mechanism of CXCR4 Antagonists on a Constitutively Active Mutant. Cell Chem Biol 26, 662-673 e667 (2019).
16. C. E. Vickers, S. F. Bydder, Y. Zhou, L. K. Nielsen, Dual gene expression cassette vectors with antibiotic selection markers for engineering in Saccharomyces cerevisiae. Microbial Cell Factories 12 (2013).
17. C. McMahon et al., Yeast surface display platform for rapid discovery of conformationally selective nanobodies. Nat Struct Mol Biol 25, 289-296 (2018).
18. C. Camacho et al., BLAST+: architecture and applications. BMC Bioinformatics 10, 421 (2009).
19. J. M. Dana et al., SIFTS: updated Structure Integration with Function, Taxonomy and Sequences resource allows 40-fold increase in coverage of structure-based annotations for proteins. Nucleic Acids Res 47, D482-D489 (2019).
20. Y. Zhang, J. Skolnick, TM-align: a protein structure alignment algorithm based on the TM-score. Nucleic Acids Res 33, 2302-2309 (2005).
21. Schrodinger, LLC (2022) The PyMOL Molecular Graphics System, Version 2.4.2.
22. D. G. Isom, H. G. Dohlman, Buried ionizable networks are an ancient hallmark of G protein-coupled receptor activation. Proc Natl Acad Sci USA 112, 5702-5707 (2015).
23. D. G. Isom, V. Sridharan, H. G. Dohlman, Regulation of Ras Paralog Thermostability by Networks of Buried Ionizable Groups. Biochemistry 55, 534-542 (2016).
24. J. B. Rowe, N. J. Kapolka, G. J. Taghon, W. M. Morgan, D. G. Isom, The evolution and mechanism of GPCR proton sensing. J Biol Chem 296, 100167 (2021).
25. D. G. Isom et al., Protons as second messenger regulators of G protein signaling. Mol Cell 51, 531-538 (2013).

We claim:

1. A yeast periplasmic ligand-trapping yeast display system comprising: a plurality of yeast cells, wherein each cell comprises:
a recombinant expression construct encoding:
a ligand;
a cell-wall anchoring protein, wherein the ligand is fused to the cell-wall anchoring protein; and
a signal sequence, wherein the signal sequence promotes secretion of the one or more ligands to the yeast cell periplasmic space; and
an inducible reporter.

2. The system of claim 1, wherein the cell-wall anchoring protein comprises an N terminus projected in the periplasmic space, and wherein the ligand is fused to the N terminus of the cell-wall anchoring protein.

3. The system of claim 1, wherein the yeast cells are Saccharomyces cerevisiae.

4. The system of claim 1, wherein the ligand is a genetically encodable peptide, protein, or nanobody.

5. The system of claim 1, wherein the signal sequence comprises a sequence at least 80% identical to SEQ ID NO:20, SEQ ID NO:21, SEQ ID NO:22, SEQ ID NO:23, SEQ ID NO:24, SEQ ID NO:25, or SEQ ID NO:26.

6. The system of claim 5, wherein the signal sequence comprises SEQ ID NO:21.

7. The system of claim 1, wherein the cell-wall anchoring protein comprises a sequence at least 80% identical to SEQ ID NO:13, SEQ ID NO:14, SEQ ID NO:15, SEQ ID NO:16, SEQ ID NO:17, SEQ ID NO:18, or SEQ ID NO:19.

8. The system of claim 7, wherein the cell-wall anchoring protein comprises SEQ ID NO:13.

9. The system of claim 1, wherein the inducible reporter produces a fluorescent protein.

10. The system of claim 9, wherein the fluorescent protein is mTurqoise2.

11. The system of claim 1, wherein expression of the inducible reporter is dependent on activation of a receptor.

12. A method of screening for a ligand that modulates a receptor function, comprising:
providing the system of claim 1;
expressing the ligand fused to the cell-wall anchoring protein;
measuring the expression of the inducible reporter; and
determining if the ligand modulates a receptor function in comparison to a reference control.

13. The method of claim 12, wherein the cell-wall anchoring protein comprises an N terminus projected in the periplasmic space, and wherein the ligand is fused to the N terminus of the cell-wall anchoring protein.

14. The method of claim 12, wherein the yeast cells are Saccharomyces cerevisiae.

15. The method of claim 12, wherein the ligand is a genetically encodable peptide, protein, or nanobody.

16. The method of claim 12, wherein the signal sequence comprises a sequence at least 80% identical to SEQ ID NO:20, SEQ ID NO:21, SEQ ID NO:22, SEQ ID NO:23, SEQ ID NO:24, SEQ ID NO:25, or SEQ ID NO:26.

17. The method of claim 16, wherein the signal sequence comprises SEQ ID NO:21.

18. The method of claim 12, wherein the cell-wall anchoring protein comprises a sequence at least 80% identical to SEQ ID NO:13, SEQ ID NO:14, SEQ ID NO:15, SEQ ID NO:16, SEQ ID NO:17, SEQ ID NO:18, or SEQ ID NO:19.

19. The method of claim 18, wherein the cell-wall anchoring protein comprises SEQ ID NO:13.

20. The method of claim 12, wherein the inducible reporter comprises produces a fluorescent protein.


SEQUENCE LISTING (44 sequences)

SEQ ID NO: 1 - protein, 89 aa, synthetic construct
  REGION 1..89: note = Description of sequence: N-terminal Alpha-PrePro signal sequence
MRFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 2 - protein, 216 aa, Synthetic construct
  REGION 1..216: note = Description of sequence: C-terminal cell wall anchor containing amino acids 23-238 of the native yeast protein Ccw14
TPPACLLACVAQVGKSSSTCDSLNQVTCYCEHENSAVKKCLDSICPNNDADAAYSAFKSSCSEQNASLGDSSSSASSSASSSSKASSSTKASSSSASSSTKASSSSASSSTKASSSSAAPSSSKASSTESSSSSSSSTKAPSSEESSSTYVSSSKQASSTSEAHSSSAASSTVSQETVSSALPTSTAVISTFSEGSGNVLEAGKSVFIAAVAAMLI

SEQ ID NO: 3 - protein, 472 aa, Synthetic construct
  REGION 1..472: note = Description of sequence: hGai
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKECGLY

SEQ ID NO: 4 - protein, 472 aa, Synthetic construct
  REGION 1..472: note = Description of sequence: hGao
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKGCGLY

SEQ ID NO: 5 - protein, 472 aa, Synthetic construct
  REGION 1..472: note = Description of sequence: hGat
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKDCGLF

SEQ ID NO: 6 - protein, 472 aa, Synthetic construct
  REGION 1..472: note = Description of sequence: hGaz
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKYIGLC

SEQ ID NO: 7 - protein, 472 aa, Synthetic construct
  REGION 1..472: note = Description of sequence: hGaq
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKEYNLV

SEQ ID NO: 8 - protein, 472 aa, Synthetic construct
  REGION 1..472: note = Description of sequence: hGa14
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKEFNLV

SEQ ID NO: 9 - protein, 472 aa, Synthetic construct
  REGION 1..472: note = Description of sequence: hGa15
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKEINLL

SEQ ID NO: 10 - protein, 472 aa, Synthetic construct
  REGION 1..472: note = Description of sequence: hGa12
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKDIMLQ

SEQ ID NO: 11 - protein, 472 aa, Synthetic construct
  REGION 1..472: note = Description of sequence: hGa13
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKQLMLQ

SEQ ID NO: 12 - protein, 472 aa, Synthetic construct
  REGION 1..472: note = Description of sequence: hGas
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKQYELL

SEQ ID NO: 13 - protein, 216 aa, Synthetic construct; identical to SEQ ID NO: 2
  REGION 1..216: note = Description of sequence: Ccw1423

SEQ ID NO: 14 - protein, 320 aa, Synthetic construct
  REGION 1..320: note = Description of sequence: Sed119-338
QFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 15 - protein, 42 aa, Synthetic construct
  REGION 1..42: note = Description of sequence: Flo11496-1537
ASSMVGYSTASLEISTYAGSANSLLAGSGLSVFIASLLLAII

SEQ ID NO: 16 - protein, 1051 aa, Synthetic construct
  REGION 1..1051: note = Description of sequence: Flo525-1075
ATEACLPAGQRKSGMNINFYQYSLKDSSTYSNAAYMAYGYASKTKLGSVGGQTDISIDYNIPCVSSSGTFPCPQEDSYGNWGCKGMGACSNSQGIAYWSTDLFGFYTTPTNVTLEMTGYFLPPQTGSYTFSFATVDDSAILSVGGSIAFECCAQEQPPITSTNFTINGIKPWDGSLPDNITGTVYMYAGYYYPLKVVYSNAVSWGTLPISVELPDGTTVSDNFEGYVYSFDDDLSQSNCTIPDPSIHTTSTITTTTEPWTGTFTSTSTEMTTITDTNGQLTDETVIVIRTPTTASTITTTTEPWTGTFTSTSTEMTTVTGTNGQPTDETVIVIRTPTSEGLITTTTEPWTGTFTSTSTEMTTVTGTNGQPTDETVIVIRTPTSEGLITTTTEPWTGTFTSTSTEVTTITGTNGQPTDETVIVIRTPTSEGLITTTTEPWTGTFTSTSTEMTTVTGTNGQPTDETVIVIRTPTSEGLISTTTEPWTGTFTSTSTEVTTITGTNGQPTDETVIVIRTPTSEGLITTTTEPWTGTFTSTSTEMTTVTGTNGQPTDETVIVIRTPTSEGLITRTTEPWTGTFTSTSTEVTTITGTNGQPTDETVIVIRTPTTAISSSLSSSSGQITSSITSSRPIITPFYPSNGTSVISSSVISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHSSSVVSVSETGNTMSLTSSGLSTMSQQPRSTPASSMVGSSTASLEISTYAGSANSLLAGSGLSVFIASLLLAII

SEQ ID NO: 17 - protein, 513 aa, Synthetic construct
  REGION 1..513: note = Description of sequence: Suc220-532
SMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 18 - protein, 410 aa, Synthetic construct
  REGION 1..410: note = Description of sequence: Ecm3320-429
ANSTTSIPSSCSIGTSATATAQADLDKISGCSTIVGNLTITGDLGSAALASIQEIDGSLTIFNSSSLSSFSADSIKKITGDLNMQELIILTSASFGSLQEVDSINMVTLPAISTFSTDLQNANNIIVSDTTLESVEGFSTLKKVNVFNINNNRYLNSFQSSLESVSDSLQFSSNGDNTTLAFDNLVWANNITLRDVNSISFGSLQTVNASLGFINNTLPSLNLTQLSKVGQSLSIVSNDELSKAAFSNLTTVGGGFIIANNTQLKVIDGFNKVQTVGGAIEVTGNFSTLDLSSLKSVRGGANFDSSSSNFSCNALKKLQSNGAIQGDSFVCKNGATSTSVKLSSTSTESSKSSATSSASSSGDASNAQANVSASASSSSSSSKKSKGAAPELVPATSFMGVVAAVGVALL

SEQ ID NO: 19 - protein, 548 aa, Synthetic construct
  REGION 1..548: note = Description of sequence: Yps122-569
KIIPAANKRDDDSNSKFVKLPFHKLYGDSLENVGSDKKPEVRLLKRADGYEEIIITNQQSFYSVDLEVGTPPQNVTVLVDTGSSDLWIMGSDNPYCSSNSMGSSRRRVIDKRDDSSSGGSLINDINPFGWLTGTGSAIGPTATGLGGGSGTATQSVPASEATMDCQQYGTFSTSGSSTFRSNNTYFSISYGDGTFASGTFGTDVLDLSDLNVTGLSFAVANETNSTMGVLGIGLPELEVTYSGSTASHSGKAYKYDNFPIVLKNSGAIKSNTYSLYLNDSDAMHGTILFGAVDHSKYTGTLYTIPIVNTLSASGFSSPIQFDVTINGIGISDSGSSNKTLTTTKIPALLDSGTTLTYLPQTVVSMIATELGAQYSSRIGYYVLDCPSDDSMEIVFDFGGFHINAPLSSFILSTGTTCLLGIIPTSDDTGTILGDSFLTNAYVVYDLENLEISMAQARYNTTSENIEIITSSVPSAVKAPGYTNTWSTSASIVTGGNIFTVNSSQTASFSGNLTTSTASATSTSSKRNVGDHIVPSLPLTLISLLFAFI

SEQ ID NO: 20 - protein, 19 aa, Synthetic construct
  REGION 1..19: note = Description of sequence: aPrenat
MRFPSIFTAVLFAASSALA

SEQ ID NO: 21 - protein, 89 aa, Synthetic construct; identical to SEQ ID NO: 1
  REGION 1..89: note = Description of sequence: aPreProv

SEQ ID NO: 22 - protein, 91 aa, Synthetic construct
  REGION 1..91: note = Description of sequence: aPreProv3
MRFPSIFTDVLFAASSALATPVNTTTEDETAQIPAEAVIGYSDLEGDFDVAVLPFSNSTNNGLLFINTTIASIAAKEEGVSLEKREAEAEF

SEQ ID NO: 23 - protein, 91 aa, Synthetic construct
  REGION 1..91: note = Description of sequence: aPreProv3
MRFPSIFTDVLFAASSALATPVNTTTEDETAQIPAEAVIGYSDLEGDFDVAVLPFSNSTNNGLLFINTTIASIAAKEEGVSLEKRETEAEF

SEQ ID NO: 24 - protein, 89 aa, Synthetic construct
  REGION 1..89: note = Description of sequence: aPreProv4
MRFPSIFTAVLFAASSALAAPANTTTEDETAQIPAEAVIDYSDLEGDFDAAALPLSNSTNNGLSSTNTTIASIAAKEEGVSLDKREAEA

SEQ ID NO: 25 - protein, 21 aa, Synthetic construct
  REGION 1..21: note = Description of sequence:
MRQVWFSWIVGFLCFFNVSSA

SEQ ID NO: 26 - protein, 91 aa, Synthetic construct
  REGION 1..91: note = Description of sequence: Ost1
MRQVWFSWIVGFLCFFNVSSAAPVNTTTEDETAQIPAEAVIGYLDLEGDFDVAVLPFSNSTNNGLLFINTTIASIAAKEEGVSLDKREAEA

SEQ ID NO: 27 - protein, 5 aa, synthetic construct
  REGION 1..5: note = Description of sequence: unique protein sequence
ECGLY

SEQ ID NO: 28 - protein, 5 aa, synthetic construct
  REGION 1..5: note = Description of sequence: unique protein sequence
GCGLY

SEQ ID NO: 29 - protein, 5 aa, synthetic construct
  REGION 1..5: note = Description of sequence: unique protein sequence
DCGLF

SEQ ID NO: 30 - protein, 5 aa, synthetic construct
  REGION 1..5: note = Description of sequence: unique protein sequence
YIGLC

SEQ ID NO: 31 - protein, 5 aa, synthetic construct
  REGION 1..5: note = Description of sequence: unique protein sequence
EYNLV

SEQ ID NO: 32 - protein, 5 aa, synthetic construct
  REGION 1..5: note = Description of sequence: unique protein sequence
EFNLV

SEQ ID NO: 33 - protein, 5 aa, synthetic construct
  REGION 1..5: note = Description of sequence: unique protein sequence
EINLL

SEQ ID NO: 34 - protein, 5 aa, synthetic construct
  REGION 1..5: note = Description of sequence: unique protein sequence
DIMLQ

SEQ ID NO: 35 - protein, 5 aa, synthetic construct
  REGION 1..5: note = Description of sequence: unique protein sequence
QLMLQ

SEQ ID NO: 36 - protein, 5 aa, synthetic construct
  REGION 1..5: note = Description of sequence: unique protein sequence
QYELL

SEQ ID NO: 37 - protein, 8 aa, synthetic construct
  REGION 1..8: note = Description of sequence: Consensus sequence
  VARIANT 2: note = X is any standard amino acid
  VARIANT 6..7: note = X is any standard amino acid
LXLSCXXS

SEQ ID NO: 38 - protein, 9 aa, synthetic construct
  REGION 1..9: note = Description of sequence: Consensus sequence
  VARIANT 1..4: note = X is any standard amino acid
  VARIANT 6: note = X is any standard amino acid
XXXXWXRQA

SEQ ID NO: 39 - protein, 6 aa, synthetic construct
  REGION 1..6: note = Description of sequence: Consensus sequence
  VARIANT 4: note = X is any standard amino acid
  VARIANT 6: note = X is any standard amino acid
RFTXSX

SEQ ID NO: 40 - protein, 9 aa, synthetic construct
  REGION 1..9: note = Description of sequence: Consensus sequence
  VARIANT 3..4: note = X is any standard amino acid
  VARIANT 6: note = X is any standard amino acid
  VARIANT 8..9: note = X is any standard amino acid
DTXXYXCXX

SEQ ID NO: 41 - protein, 6 aa, synthetic construct
  REGION 1..6: note = Description of sequence: Consensus sequence
  VARIANT 4..5: note = X is any standard amino acid
LSCXXS

SEQ ID NO: 42 - protein, 5 aa, synthetic construct
  REGION 1..5: note = Description of sequence: identifying peptide subsequence
SISSI

SEQ ID NO: 43 - protein, 5 aa, synthetic construct
  REGION 1..5: note = Description of sequence: identifying peptide subsequence
NIFDV

SEQ ID NO: 44 - protein, 5 aa, synthetic construct
  REGION 1..5: note = Description of sequence: identifying peptide subsequence
SIFSI